SEQUENCE LISTING (1 sequence)

SEQ ID NO: 1 - protein, 43 aa, Homo sapiens
MDAEFRHDSGYEVHHQKLVFFAEDVGSNKGAIIGLMVGGVVIA

(19) United States
(12) Patent Application Publication
(10) Pub. No.: US 2021/0145801 A1
(43) Pub. Date: May 20, 2021

(54) THERAPY FOR PROTEIN MISFOLDING DISEASE

(71) Applicant: WREN THERAPEUTICS LIMITED, Cambridge (GB)

(72) Inventors: Johnny HABCHI, Cambridge (GB); Xiaoting YANG, Cambridge (GB); Kerry JENKINS, Cambridge (GB); Michele PERNI, Cambridge (GB); Sunehera SARWAT, Cambridge (GB); Joseph MENZIES, Cambridge (GB); Cristina CAMPERO PEREDO, Valle de Bravo (MX); Andrea POSSENTI, Cambridge (GB); Sara LINSE, Cambridge (GB); Tuomas KNOWLES, Cambridge (GB); Christopher DOBSON, Cambridge (GB); Samuel COHEN, Cambridge (GB); Michele VENDRUSCOLO, Cambridge (GB)

(21) Appl. No.: 17/045,075

(22) PCT Filed: Apr. 4, 2019
(86) PCT No.: PCT/GB2019/050982

§ 371 Date: Oct. 2, 2020

(30) Foreign Application Priority Data
Apr. 4, 2018 (GB) 1805554.1

(51) Int. Cl. A61K 31/426; A61P 25/28

(52) U.S. Cl. CPC A61K 31/426; A61P 25/28

(57) ABSTRACT

A thiazolidinedione or rhodanine compound or a pharmaceutically acceptable salt, tautomer, solvate, hydrate, prodrug, derivative, stereoisomer, analog or isotopically labelled derivative thereof, for use in the treatment and/or prevention of a protein misfolding disease, wherein said compound is not Pioglitazone, Rosiglitazone, Rivoglitazone, Balaglitazone or Mitoglitazone.

### FIELD OF THE INVENTION

The present invention relates to novel therapies for the treatment and/or prevention of a protein misfolding disease and/or a neurodegenerative disease using a thiazolidinedione or rhodanine compound which is not Pioglitazone, Rosiglitazone, Rivoglitazone, Balaglitazone or Mitoglitazone. In particular, the present invention is concerned with compounds of formula (I) as novel therapies for the treatment and/or prevention of Alzheimer's disease (AD) and other diseases which may be associated with or caused by misfolding of the amyloid-β peptide.

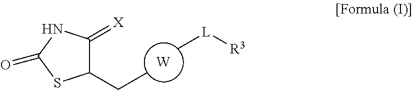

### BACKGROUND OF THE INVENTION

In protein misfolding diseases, also called proteinopathies, the abnormal folding of certain proteins disrupts normal cell function. In some cases, the abnormal protein is toxic, while in others the symptoms are caused by the loss of function of the protein. Protein misfolding or aggregation is a feature of many neurodegenerative diseases.

Neurodegenerative diseases are characterised by the loss of structure or function of neurons, including neuronal death. Neurodegenerative diseases have a range of causes, and can be found on many different levels of neuronal circuitry, from molecular to systemic. Several neurodegenerative diseases are proteinopathies, including Alzheimer's disease (AD), Parkinson's disease (PD), tauopathies, and polyglutamine expansion diseases, such as Huntington's disease. At present, there are no effective strategies to slow, prevent or treat the neurodegeneration associated with these diseases in humans.

The incidence of AD is increasing rapidly as the global population ages. The characteristic deterioration of cognitive abilities affects memory, language skills and self-care, and AD often causes severe disruption to the patient's lifestyle and independence. AD affects over 35 million people worldwide, a figure that is expected to rise to 115 million by 2050. The condition thus represents a significant burden for healthcare systems, with estimated costs approaching a trillion euros worldwide.

AD is still incurable, as no disease-modifying drug is currently available on the market and over 400 clinical trials for this disease have already failed. Successful trials are thus needed to deliver new treatments to AD patients.

The amyloid-β (Aβ) peptide is widely considered to play a central role in AD. The Aβ peptide is produced in different isoforms and self-assembles into neurotoxic aggregates and forms the amyloid deposits that are found post mortem in the brains of AD patients. Mainly, the 40- and 42-residue isoforms are found in the brain of AD patients. Several amyloid targeted strategies have been pursued in the past decades, including decreasing Aβ production, modulating Aβ transport, increasing Aβ clearance and decreasing Aβ aggregation. However, so far such strategies have not brought an effective drug to market. It is particularly desirable to develop inhibition strategies based on the use of drugs already validated for the treatment of other conditions or compounds known to be pharmaceutically acceptable.

The present inventors have used a high-throughput kinetics-based screening of libraries to identify inhibitors of Aβ aggregation, based on their ability to inhibit specific microscopic processes in the Aβ aggregation process which result in the reduction of the population(s) of toxic oligomeric aggregates. The libraries that have been screened consisted of drugs that have been approved by regulatory authorities (such as FDA, EMA, PMDA and others) in addition to experimental drugs that have entered clinical trials but have not been approved by any regulatory authority,

### SUMMARY

Surprisingly, a series of thiazolidinedione compounds, including Netoglitazone, were found to be excellent inhibitors of Aβ aggregate formation. The present invention therefore provides a thiazolidinedione or rhodanine compound which is not Pioglitazone, Rosiglitazone, Rivoglitazone, Balaglitazone or Mitoglitazone, or a pharmaceutically acceptable salt, tautomer, solvate, hydrate, prodrug, derivative, stereoisomer, analog or isotopically labelled derivative thereof, for use in the treatment and/or prevention of a protein misfolding disease.

The present invention also provides a thiazolidinedione or rhodanine compound comprising, at opposite ends of the molecule, a primary terminal group which is a thiazolidinedione or rhodanine group and a secondary terminal group which is not (i) a 5- to 10-membered partially unsaturated heterocyclyl group containing one or more nitrogen heteroatoms in the ring, or (ii) a 5- to 10-membered heteroaryl group containing one or more nitrogen heteroatoms in the ring, or a pharmaceutically acceptable salt, tautomer, solvate, hydrate, prodrug, derivative, stereoisomer, analog or isotopically labelled derivative thereof, for use in the treatment and/or prevention of a protein misfolding disease.

The present invention further provides a compound for use as described above, wherein the compound is a compound of formula (I), or a pharmaceutically acceptable salt, tautomer, solvate, hydrate, prodrug, derivative, stereoisomer, analog or isotopically labelled derivative thereof,

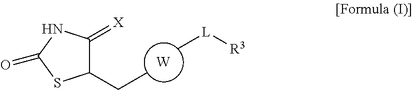

wherein X represents O or S, W represents a benzene, naphthalene, benzodihydropyran or benzopyran ring, which is optionally further substituted, L represents a linker group which comprises an alkylene group optionally comprising (i) one or more heteroatoms and/or carbonyl groups; and/or (ii) a 5- to 10-membered saturated or unsaturated heterocyclic group which is optionally substituted and R³ represents an optionally substituted C₆ to C₁₀ aryl group, C₅ to C₉ carbocyclyl group, 5- to 9-membered saturated heterocyclyl group, 5- to 9-membered partially unsaturated heterocyclyl group which does not contain a nitrogen heteroatom in the ring, or a 5- to 10-membered heteroaryl group which does not contain a nitrogen heteroatom in the ring.

In another embodiment of the invention, the compound is a compound of formula (IA), or a pharmaceutically acceptable salt, tautomer, solvate, hydrate, prodrug, derivative, stereoisomer, analog or isotopically labelled derivative thereof,

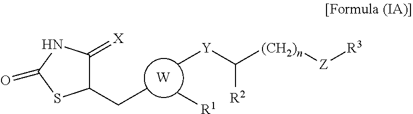

wherein X represents O or S, W represents a benzene or naphthalene ring, which is optionally further substituted, Y represents O or a carbonyl C(O) group, R₁ and R₂ are the same or different and each independently represent hydrogen or a substituted or unsubstituted C₁ to C₄ alkyl group or are linked to form a 5 to 7 membered aryl, carbocyclyl or heterocyclyl ring, which is optionally further substituted, n is an integer of from 0 to 2, Z represents a bond or a 5- to 10-membered saturated or unsaturated heterocyclic group which is optionally substituted, and R³ represents an optionally substituted C₆ to C₁₀ aryl group, optionally substituted C₅ to C₁₀ carbocyclyl group, or an optionally substituted heterocyclyl group selected from pyranyl, dihydropyranyl, dihydrofuranyl, dihydrobenzofuranyl, dihydroisobenzofuranyl, benzopyranyl, dihydrobenzopyranyl, furanyl and benzofuranyl.

Preferably, X represents O, W represents a benzene or naphthalene ring, Y represents O, R¹ and R² each independently represent hydrogen or are linked to form, together with W, a benzopyran or benzodihydropyran ring, and n is 0 or 1.

In another embodiment of the invention, the compound is a compound of formula (II) or (III), or a pharmaceutically acceptable salt, tautomer, solvate, hydrate, prodrug, derivative, stereoisomer, analog or isotopically labelled derivative thereof,

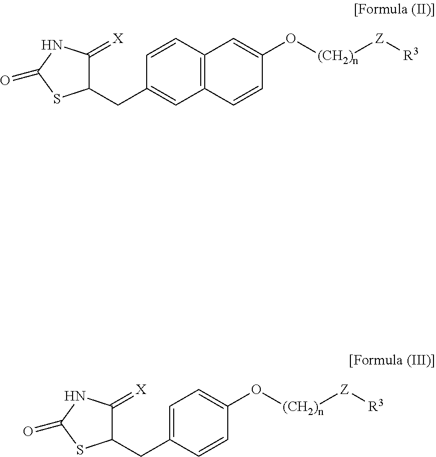

wherein n is 1 or 2 and the other chemical groups are as defined above.

Preferably, Z represents a bond.

Preferably, X represents oxygen.

Preferably, R³ represents an optionally substituted C₆ to C₁₀ aryl group or an optionally substituted C₅ to C₁₀ carbocyclyl group.

In a further embodiment of the invention, the compound is Netoglitazone, Ciglitazone, Englitazone, Darglitazone or Troglitazone, or a pharmaceutically acceptable salt, tautomer, solvate, hydrate, prodrug, derivative, stereoisomer, analog or isotopically labelled derivative thereof.

In a preferred embodiment, the compound is Netoglitazone or a pharmaceutically acceptable salt, tautomer, solvate, hydrate, prodrug, derivative, stereoisomer, analog or isotopically labelled derivative thereof.

In one embodiment, the compound of the invention as defined above is for use in treating, preventing or inhibiting the formation, deposition, accumulation, or persistence of oligomers, fibrils, aggregates and/or plaques of proteins and/or peptides.

Preferably, the compound of the invention is for use in treating, preventing or inhibiting the formation, deposition, accumulation, or persistence of amyloid β peptide oligomers, fibrils, aggregates and/or plaques.

Preferably, the compound of the invention is for use in treating a protein misfolding disease which is associated with misfolding of the amyloid β peptide.

Preferably, the protein misfolding disease is selected from amyloidosis, tauopathies, prion diseases (including Creutzfeld-Jakob disease and spongiform encephalopathies), neurodegenerative disease, Down syndrome, and/or cystic fibrosis.

More preferably, the protein misfolding disease is a neurodegenerative disease.

Even more preferably, the neurodegenerative disease is selected from dementia, mild cognitive impairment (MCI), Parkinson's disease, polyglutamine diseases (such as Huntington's disease) and/or amyotrophic lateral sclerosis (ALS).

Preferably, the dementia is selected from Alzheimer's disease, dementia with Lewy Bodies, frontotemporal dementia, familial dementia and/or progressive supranuclear palsy (PSP).

In another embodiment, the protein misfolding disease is selected from Alzheimer's disease, cerebral amyloid-β angiopathy, inclusion body myositis and/or Down's syndrome.

Most preferably, the protein misfolding disease is Alzheimer's disease.

In another embodiment of the invention, the compound of the invention as defined above or a pharmaceutically acceptable salt, tautomer, solvate, hydrate, prodrug, derivative, stereoisomer, analog or isotopically labelled derivative thereof is for use in the treatment or prevention of a neurodegenerative disease.

Preferably, the neurodegenerative disease is selected from dementia, mild cognitive impairment (MCI), Parkinson's disease, polyglutamine diseases (such as Huntington's disease) and/or amyotrophic lateral sclerosis (ALS).

More preferably, the dementia is selected from Alzheimer's disease, dementia with Lewy Bodies, frontotemporal dementia, familial dementia and/or progressive supranuclear palsy (PSP).

Most preferably, the dementia is Alzheimer's disease.

When the compound of the invention as defined above is for use in the treatment and/or prevention of Alzheimer's disease, the Alzheimer's disease is preferably stage one, stage two or stage three Alzheimer's disease according to the Reisberg scale.

In an embodiment of the invention, the compound of the invention is for use in the treatment of a patient which has been diagnosed with, or is at risk of developing, Alzheimer's disease.

In one embodiment, the patient has been diagnosed with mild cognitive impairment (MCI).

In one embodiment, the patient has a family history of Alzheimer's disease.

The present invention also provides a pharmaceutical composition comprising the compound of the invention as defined above or a pharmaceutically acceptable salt, tautomer, solvate, hydrate, prodrug, derivative, stereoisomer, analog or isotopically labelled derivative thereof, for use in the treatment and/or prevention of a protein misfolding disease and/or a neurodegenerative disease.

Preferably, the pharmaceutical composition is for use in the treatment and/or prevention of a protein misfolding disease and/or a neurodegenerative disease as defined above.

In one embodiment, the pharmaceutical composition further comprises one or more additional pharmaceutically active agents.

In another embodiment, the additional pharmaceutically active agent(s) are suitable for the treatment and/or prevention of a protein misfolding disease and/or a neurodegenerative disease.

In a further embodiment, the compound of the invention and the additional pharmaceutically active agent(s) are formulated for separate, concurrent, simultaneous or successive administration.

Preferably, the pharmaceutical composition of the invention is formulated to improve penetration of the compound as described above into the brain. More preferably, the pharmaceutical composition comprises nanoparticle carriers based on polymers, lipids, protein capsules or combinations thereof.

The present invention also provides a kit comprising the compound of the invention as defined above or a pharmaceutically acceptable salt, tautomer, solvate, hydrate, prodrug, derivative, stereoisomer, analog or isotopically labelled derivative thereof, or the composition of the invention as defined above, for use in the treatment and/or prevention of a protein misfolding disease and/or a neurodegenerative disease. Optionally, the kit further comprises, in admixture or in separate containers, an additional pharmaceutically active agent(s) as defined above.

The present invention additionally provides a method of treating and/or preventing a protein misfolding disease and/or a neurodegenerative disease in a patient which comprises administering to said patient an effective amount of the compound of the invention as defined above or a pharmaceutically acceptable salt, tautomer, solvate, hydrate, prodrug, derivative, stereoisomer, analog or isotopically labelled derivative thereof. Preferably, the protein misfolding disease and/or neurodegenerative disease is as defined above. Most preferably, the protein misfolding disease and/or neurodegenerative disease is Alzheimer's disease.

The present invention further provides the use of the compound of the invention as defined above or a pharmaceutically acceptable salt, tautomer, solvate, hydrate, prodrug, derivative, stereoisomer, analog or isotopically labelled derivative thereof in the manufacture of a medicament for the treatment and/or prevention of a protein misfolding disease and/or a neurodegenerative disease. Preferably, the protein misfolding disease and/or neurodegenerative disease is as defined above. Most preferably, the protein misfolding disease and/or neurodegenerative disease is Alzheimer's disease.

### BRIEF DESCRIPTION OF THE DRAWINGS

FIG. 1—Netoglitazone inhibits Aβ aggregation. (a) Normalised kinetic profiles of the aggregation of a 2 μM solution of Aβ42 in the absence and presence of a range of Netoglitazone concentrations, shown using different symbols. (b) Relative half-times of the aggregation course reactions, with respect to DMSO, derived from (a) as a function of Netoglitazone concentration. (c) Comparative time course of the formation of 2 μM Aβ42 fibrils in the absence and presence of 5-fold excess of Netoglitazone using a dot-blot assay. (d-e) Characterization of the effects of Netoglitazone on Aβ42 aggregation using quantitative chemical kinetics. The abbreviation kₙ is the rate constant for primary nucleation, k₊ is the rate constant for elongation, and k₂ is the rate constant for secondary nucleation. Only predictions when both primary and secondary pathways are inhibited fit the experimental data well. The dependence of the apparent reaction rate constants (k^{app}) of primary pathways (d, kₙk₊), and secondary pathways (e, k₂k₊), is shown with increasing concentrations of Netoglitazone relative to the values in the absence of Netoglitazone. In each case, k represents either kₙk₊ (primary pathways) or k₂k₊ (secondary pathways). (f-i) Characterization of the effects of Netoglitazone on the secondary pathways of Aβ42 aggregation. (f) Normalised kinetic aggregation profiles of a 2 μM Aβ42 solution in the absence and the presence of 2% and 50% of preformed seeds. (g) Normalised kinetic aggregation profiles of a 2 μM Aβ42 solution in the presence of 50% of preformed seeds in the absence and presence of a range of concentrations of Netoglitazone. Under these conditions, elongation of the fibrils is the dominant mechanism; these results show that Netoglitazone, at concentrations as high as 20-fold excess, does not affect the elongation rates of Aβ42 aggregation. (h) Normalised kinetic aggregation profiles of a 2 μM Aβ42 solution in the presence of 2% of preformed seeds in the absence and presence of a range of Netoglitazone concentrations. (i) Effect of Netoglitazone on the rate constant of the surface-catalyzed secondary nucleation (k₂). The rate constants were obtained from the aggregation kinetics of a 2 μM Aβ42 solution in the presence of 2% of preformed seeds, where primary nucleation is negligible. The observed effects could only be due to decreasing the rate constants of surface-catalyzed secondary nucleation because elongation is not affected by the compounds under these conditions. (j) Effect of Netoglitazone on Aβ42 aggregation in 66% CSF. Normalised kinetic profiles of the aggregation of a 2 μM Aβ42 solution in the absence and presence of a range of Netoglitazone concentrations. (k) Effect of Netoglitazone on Aβ40 aggregation. Normalised kinetic profiles of the aggregation of a 10 μM Aβ40 solution in the absence and presence of 1.25-fold excess of Netoglitazone. (l-o) Effect of Netoglitazone on Aβ42 oligomer production and resulting toxicity. (1) Normalised kinetic profiles of the aggregation of a 2 μM Aβ42 solution in the absence and presence of 5-fold excess of Netoglitazone. (m) Simulated time evolution of the nucleation rates corresponding to the reactions in (1). (n) Quantification of the peak time and peak area from (m) in the absence and the presence of 5-fold excess of Netoglitazone. (o) Effect of Netoglitazone on the disruption of lipid membranes by Aβ42 oligomers. 5-fold excess of Netoglitazone decreased substantially the toxic effect from a 2 μM Aβ42 solution in disrupting lipid vesicles measured at the half-time of the aggregation reaction of Aβ42 alone. Bars represent the resulting fluorescence from the binding of a fluorescence dye contained in the vesicles to Ca²⁺ present outside of the vesicles as a result of the influx of Ca²⁺ in the presence of oligomers. The bar labelled Aβ monomer is a measurement from a 2 μM Aβ42 solution at time 0 h in the absence of Netoglitazone; the bar labelled DMSO is a measurement from a 2 μM Aβ42 solution at time 2 h in the absence of Netoglitazone; the bar labelled 5-fold excess is a measurement from a 2 μM Aβ42 solution at time 2 h where 5-fold excess of Netoglitazone was added to the Aβ42 solution at time 0 h. (p) Measurement of the concentrations of Aβ42 oligomers in the absence and presence of Netoglitazone. ELISA of 5 μM Aβ42 alone or 5 μM Aβ42 in the presence of 5-fold excess of Netoglitazone at the half-time of the aggregation reaction of Aβ42 alone. The bar-plot shows the relative concentrations of oligomers measured using an oligomer-specific antibody.

FIG. 2—Netoglitazone protects AD worms (GMC101) from Aβ-associated toxicity. (a) Netoglitazone was administered to C. elegans at the larval stage L4 to mimic a preventive strategy and also at a later stage (D3 of adulthood) to mimic a therapeutic intervention. (b) Administration of increasing concentrations (0, 0.05, 0.1, 0.5, 5, 10 μM) of Netoglitazone to C. elegans models of AD (left), Control healthy animals (centre), and worm models of PD (right) leads to a dose dependent and statistically significant recovery in the dysfunctional phenotype with high specificity. The effect is maximum between 0.5 and 5 μM. (c) Representative pictures showing the movement over 5 s of AD, Control and Netoglitazone treated animals. White arrows indicate paralyzed animals. The treatment greatly improves the mobility of the AD worms. (d-e) Decrease in the plaques load in AD worms at day 6 of adulthood following the treatment with Netoglitazone at L4. Quantification (left) of fluorescence intensity and representative images (right) of treated and untreated AD worms. (f) Administration of Netoglitazone at L4 restores the ROS production in AD worms to normal levels at D5 of adulthood. (g) The maximum tolerable dose for Netoglitazone appears to be less than 50 μM (left panel) and 500 μM (right panel) in AD and control animals, respectively. (h-j) Netoglitazone late administration (D3) decreases the plaques load at D6 of adulthood (h) and improves motility (i) and survival rates (j) at D5 of adulthood in AD animals.

FIG. 3—Normalised kinetic profiles of the aggregation of a 2 μM solution of Aβ42 in the absence and presence of Netoglitazone, Ciglitazone, Englitazone, Darglitazone and Troglitazone at 5× drug:protein concentration.

FIG. 4—Normalised kinetic profiles of the aggregation of a 2 μM solution of Aβ42 in the absence and presence of Pioglitazone, Rosiglitazone, Rivoglitazone, Balaglitazone and Mitoglitazone at 5× drug:protein concentration.

FIG. 5—Relative half-times of the aggregation course reactions, with respect to DMSO, derived from FIGS. 3 and 4.

FIG. 6—Chemotaxis and motility measurements showing the effects of Netoglitazone on additional AD worm models. (A-B) Netoglitazone significantly improves the (A) chemotaxis index and (B) motility of Aβ_{1-42Neur} worms when compared to control wild type worms. (C) General chemotaxis experimental diagram (O. Margie, C. Palmer, I. Chin-Sang, C. elegans Chemotaxis Assay. J Vis Exp, e50069 (2013)). Worms are positioned in the centre of the plate while the attractants are positioned in two quadrants. After 8 h the CI index is calculated. Healthy worms are expected to move to the quadrants containing attractants (A and B) and avoid the test quadrants (C and D). (D) Netoglitazone significantly improves the motility of Aβ₃₋₄₂::GF_{PMuscular} worms. Errors represent the Standard Error on the Mean (SEM). For the above experiments ca. 200 worms were used in (A) and ca. 600 worms were used in (B,D). For statistical significance tests, a one-way ANOVA was carried out using GraphPadPrism.

FIG. 7—Data relating to pharmacokinetics studies in mice. (a) Pharmacokinetic time course of Netoglitazone (11.5 mg/kg, p.o.) in male Swiss Albino mice. Matrix: Blood plasma, solid line/open circles; brain homogenate, dotted line/filled diamonds; CSF, dashed line/filled squares. (b) Histogram of Netoglitazone microdialysate levels expressed as ng/ml corrected for probe recovery (0.11). Arrow denotes time of PO administration.

FIG. 8—Data relating to Aβ plaque detection studies in mice. (A-B) Example of Aβ plaque detection performed by the algorithm used in the mouse brain data analysis, with (A) panels showing a log-scale visualization (for easier visual comparison) of two planes from the left and the right hemisphere of a mouse brain, respectively, and (B) panels showing the plaques detected by the algorithm on the two planes shown in panel (A), with the total number of plaques reported on top of each figure. The values shown along the left and bottom of each panel indicate numbers of pixels. (C) Relative number and area of Aβ plaques in mouse brains following 90 days of once daily treatment with either placebo or Netoglitazone (75 mg/kg/day). Animals were treated from 60 days of age. N=4 mice per group; all males. Percentage number and area of plaques relative to the placebo group are expressed as mean+SEM.

### DETAILED DESCRIPTION OF THE INVENTION

### Definitions

As used herein, the term “patient” typically refers to a human patient. Patients may, however, be other vertebrate animals, such as mammals. The terms “subject” and “patient” are used interchangeably herein.

As used herein, the words “treatment” and “treating” are to be understood as embracing treatment and/or amelioration and/or prevention of or reduction in aggravation/worsening of symptoms of a disease or condition as well as treatment of the cause of the disease or condition, and may include reversing, reducing, or arresting the symptoms, clinical signs, and underlying pathology of a condition in a manner to improve or stabilise a subject's condition.

Reference to “prevention” and “preventing” a disease or condition embraces prophylaxis and/or inhibition of the disease or condition. The term “preventing” is art-recognized, and when used in relation to a condition, such as Alzheimer's disease (AD) or its associated symptoms, is well understood in the art, and includes administration of a drug and/or composition which reduces the frequency of, or delays the onset of, symptoms of a medical condition in a subject relative to a subject which does not receive the drug or composition.

As used herein, the term “pharmaceutically acceptable” refers to a material that does not interfere with the effectiveness of the compound of the invention and is compatible with a biological system such as a cell, cell culture, tissue, or organism. Preferably, the biological system is a living organism, such as a vertebrate.

As used herein, the phrase “therapeutically effective amount” refers to an amount of a compound, material or composition that is effective for producing some desired therapeutic effect, such as treating, preventing or ameliorating a protein misfolding disease or reducing the prevalence of misfolded protein, at a reasonable benefit/risk ratio applicable to any medical treatment. In one embodiment, the therapeutically effective amount is sufficient to reduce or eliminate at least one symptom. A therapeutically effective amount may partially improve a disease or symptom without fully eradicating the disease or symptom.

Compounds

The compound for use in the present invention is a thiazolidinedione or rhodanine compound which is not Pioglitazone, Rosiglitazone, Rivoglitazone, Balaglitazone or Mitoglitazone.

In particular, the compound of the invention may be a thiazolidinedione or rhodanine compound comprising, at opposite ends of the molecule, a primary terminal group which is a thiazolidinedione or rhodanine group and a secondary terminal group which is not (i) a 5- to 10-membered partially unsaturated heterocyclyl group containing one or more nitrogen heteroatoms in the ring, or (ii) a 5- to 10-membered heteroaryl group containing one or more nitrogen heteroatoms in the ring.

In one embodiment, the compound of the invention is a compound of formula (I).

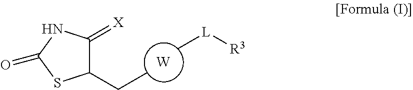

In the compound of the invention, X represents O or S. Preferably X is O.

In the compound of the invention, W represents an optionally further substituted benzene, naphthalene, benzodihydropyran or benzopyran ring, preferably an optionally further substituted benzene or naphthalene ring, more preferably an unsubstituted benzene or naphthalene ring. In one embodiment, W represents an unsubstituted naphthalene ring.

In the compound of the invention, L represents a linker group which comprises an alkylene group optionally comprising (i) one or more heteroatoms and/or carbonyl groups; and/or (ii) a 5- to 10-membered saturated or unsaturated heterocyclic group which is optionally substituted. In particular, L may represent an alkylene group optionally comprising (i) one or more heteroatoms and/or carbonyl groups; and/or (ii) a 5- to 10-membered saturated or unsaturated heterocyclic group which is optionally substituted. Preferably the heteroatom is an oxy ether group or a secondary amino group which is optionally further substituted, for example by a C₁ to C₄ alkylene group. In one embodiment, L represents a C₁ to C₄ alkylene group comprising (i) an oxy, amino and/or carbonyl group and/or (ii) a 5- to 10-membered saturated or unsaturated heterocyclic group. Preferably the heterocyclic group is an optionally substituted oxazole, isoxazole, furan, pyrrole, pyridine, pyridazine, pyrimidine or pyrazine ring. In a preferred embodiment, L represents a C₁ to C₄ alkylene group comprising: an oxy group, carbonyl group and/or an optionally substituted a 5- to 10-membered saturated or unsaturated heterocyclic group selected from an oxazole, isoxazole, furan and pyrrole ring. The optional substituent(s) of the heterocyclic group may be, for example, a halogen, —OR^{a}, —SR^{a}, —NR^{a}R^{b}, —C(O)OR^{a}, —C(O)NR^{a}R^{b}, —C(O)R^{a} and/or a C₁ to C₄ alkyl group as described further below, preferably a hydroxyl, halogen and/or C₁ to C₄ alkyl group. Preferably L represents a C₁ to C₄ alkylene group comprising an oxy and/or carbonyl group.

In the compound of the invention, R³ represents an optionally substituted C₆ to C₁₀ aryl group, optionally substituted C₅ to C₁₀ carbocyclyl group, optionally substituted 5- to 10-membered saturated heterocyclyl group, optionally substituted 5- to 10-membered partially unsaturated heterocyclyl group which does not contain a nitrogen heteroatom in the ring, or optionally substituted 5- to 10-membered heteroaryl group which does not contain a nitrogen heteroatom in the ring. Preferably, R³ represents an optionally substituted C₆ to C₁₀ aryl group, optionally substituted C₅ to C₁₀ carbocyclyl group, or an optionally substituted heterocyclyl group selected from pyranyl, dihydropyranyl, dihydrofuranyl, dihydrobenzofuranyl, dihydroisobenzofuranyl, benzopyranyl, dihydrobenzopyranyl, furanyl and benzofuranyl, The optional substituent(s) may be a halogen, —OR^{a}, —SR^{a}, —NR^{a}R^{b}, —C(O)OR^{a}, —C(O)NR^{a}R^{b}, —C(O)R^{a} and/or a C₁ to C₄ alkyl group as described further below. Preferably, R³ represents a C₆ to C₁₀ aryl group optionally substituted by one or more hydroxyl, halogen and/or C₁ to C₄ alkyl groups, a C₅ to C₁₀ carbocyclyl group optionally substituted by one or more hydroxyl, halogen and/or C₁ to C₄ alkyl groups, or a heterocyclyl group selected from pyranyl, dihydropyranyl, dihydrofuranyl, dihydrobenzofuranyl, dihydroisobenzofuranyl, benzopyranyl, dihydrobenzopyranyl, furanyl and benzofuranyl, optionally substituted by one or more hydroxyl, halogen and/or C₁ to C₄ alkyl groups. Preferably R³ represents a C₆ to Cm aryl group or a C₅ to C₁₀ carbocyclyl group which is optionally substituted by one or more hydroxyl, halogen and/or C₁ to C₄ alkyl groups, in particular a C₆ to C₁₀ aryl group optionally substituted by one or more hydroxyl, halogen and/or C₁ to C₄ alkyl groups. More preferably, R³ represents a phenyl ring optionally substituted by one or more halogen groups, in particular phenyl or fluorophenyl.

In one preferred embodiment of the compound of Formula (I):

X represents O;

W represents a benzene or naphthalene ring, optionally substituted with one or more halogen, —OR^{a}, —SR^{a}, —NR^{a}R^{b}, —C(O)OR^{a}, —C(O)NR^{a}R^{b}, —C(O)R^{a} and/or C₁ to C₄ alkyl group(s) as described further below;

L represents a C₁ to C₄ alkylene group comprising an oxy group, carbonyl group and/or an oxazole, isoxazole, furan or pyrrole ring which is optionally substituted with one or more halogen, —OR^{a}, —SR^{a}, —NR^{a}R^{b}, —C(O)OR^{a}, —C(O)NR^{a}R^{b}, —C(O)R^{a} and/or a C₁ to C₄ alkyl group(s) as described further below, preferably a hydroxyl, halogen and/or C₁ to C₄ alkyl group; and

R³ represents a C₆ to C₁₀ aryl group optionally substituted with one or more halogen, —OR^{a}, —SR^{a}, —NR^{a}R^{b}, —C(O)OR^{a}, —C(O)NR^{a}R^{b}, —C(O)R^{a} and/or C₁ to C₄ alkyl group(s) as described further below, or a C₅ to C₁₀ carbocyclyl group optionally substituted with one or more halogen, —OR^{a}, —SR^{a}, —NR^{a}R^{b}, —C(O)OR^{a}, —C(O)NR^{a}R^{b}, —C(O)R^{a} and/or C₁ to C₄ alkyl group(s) as described further below.

Preferably, in the compound of Formula (I):

X represents O;

W represents an unsubstituted benzene or naphthalene ring;

L represents a C₁ to C₄ alkylene group comprising an oxy and/or carbonyl group; and

R³ represents a C₆ to C₁₀ aryl group optionally substituted by one or more hydroxyl, halogen and/or C₁ to C₄ alkyl groups.

In one preferred embodiment, the compound of the invention may be a compound of formula (IA), wherein X, W and R³ are as defined above.

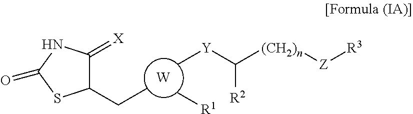

In particular, in the compound of Formula (IA), W represents an optionally further substituted benzene or naphthalene ring, more preferably an unsubstituted benzene or naphthalene ring. In one embodiment, W represents an unsubstituted naphthalene ring.

In particular, in the compound of Formula (IA), R³ represents an optionally substituted C₆ to C₁₀ aryl group, an optionally substituted C₅ to C₁₀ carbocyclyl group, or an optionally substituted heterocyclyl group selected from pyranyl, dihydropyranyl, dihydrofuranyl, dihydrobenzofuranyl, dihydroisobenzofuranyl, benzopyranyl, dihydrobenzopyranyl, furanyl and benzofuranyl. The optional substituent(s) may be a halogen, —OR^{a}, —SR^{a}, —NR^{a}R^{b}, —C(O)OR^{a}, —C(O)NR^{a}R^{b}, —C(O)R^{a} and/or a C₁ to C₄ alkyl group as described further below. Preferably, R³ represents a C₆ to C₁₀ aryl group optionally substituted by one or more hydroxyl, halogen and/or C₁ to C₄ alkyl groups, a C₅ to C₁₀ carbocyclyl group optionally substituted by one or more hydroxyl, halogen and/or C₁ to C₄ alkyl groups, or a heterocyclyl group selected from pyranyl, dihydropyranyl, dihydrofuranyl, dihydrobenzofuranyl, dihydroisobenzofuranyl, benzopyranyl, dihydrobenzopyranyl, furanyl, and benzofuranyl, optionally substituted by one or more hydroxyl, halogen and/or C₁ to C₄ alkyl groups. Preferably R³ represents a C₆ to C₁₀ aryl group or a C₅ to C₁₀ carbocyclyl group which is optionally substituted by one or more hydroxyl, halogen and/or C₁ to C₄ alkyl groups, in particular a C₆ to C₁₀ aryl group optionally substituted by one or more hydroxyl, halogen and/or C₁ to C₄ alkyl groups. More preferably, R³ represents a phenyl ring optionally substituted by one or more halogen groups, in particular phenyl or fluorophenyl.

In the compound of Formula (IA), Y represents O or a carbonyl C(O) group. Preferably Y is O.

In the compound of Formula (IA), R¹ and R² are the same or different and each independently represent hydrogen or a substituted or unsubstituted C₁ to C₄ alkyl group, or R¹ and R² are linked to form a 5 to 7 membered aryl, carbocyclyl or heterocyclyl ring, which is optionally further substituted. Preferably, R¹ and R² each independently represent hydrogen, or R¹ and R² are linked to form, together with W, a benzopyran or benzodihydropyran ring. Preferably, R¹ and R² are both hydrogen.

In the compound of Formula (IA), n is an integer of from 0 to 2. Preferably, n is 0 or 1. More preferably, n is 0.

In the compound of Formula (IA), Z represents a bond or a 5- to 10-membered saturated or unsaturated heterocyclic group which is optionally substituted. Preferably, Z represents a bond or an optionally substituted oxazole, isoxazole, furan, pyrrole, pyridine, pyridazine, pyrimidine or pyrazine ring, wherein the optional substituent is preferably one or more halogen, —OR^{a}, —SR^{a}, —NR^{a}R^{b}, —C(O)OR^{a}, —C(O)NR^{a}R^{b}, —C(O)R^{a} and/or C₁ to C₄ alkyl group(s) as described further below, preferably a hydroxyl, halogen and/or C₁ to C₄ alkyl group.

In one preferred embodiment of the compound of Formula (IA):

X represents O;

W represents a benzene or naphthalene ring, optionally substituted with one or more halogen, —OR^{a}, —SR^{a}, —NR^{a}R^{b}, —C(O)OR^{a}, —C(O)NR^{a}R^{b}, —C(O)R^{a} and/or C₁ to C₄ alkyl group(s) as described further below;

Y represents O;

R¹ and R² each independently represent hydrogen; or

R¹ and R² are linked to form, together with W, a benzopyran or benzodihydropyran ring; and

n is 0 or 1;

preferably wherein Z is a bond or an optionally substituted oxazole, isoxazole, furan or pyrrole ring, wherein the optional substituent is preferably one or more halogen, —OR^{a}, —SR^{a}, —NR^{a}R^{b}, —C(O)OR^{a}, —C(O)NR^{a}R^{b}, —C(O)R^{a} and/or C₁ to C₄ alkyl group(s) as described further below, preferably a hydroxyl, halogen and/or C₁ to C₄ alkyl group; and/or R³ represents a C₆ to C₁₀ aryl group optionally substituted with one or more halogen, —OR^{a}, —SR^{a}, —NR^{a}R^{b}, —C(O)OR^{a}, —C(O)NR^{a}R^{b}, —C(O)R^{a} and/or C₁ to C₄ alkyl group(s) as described further below, a C₅ to C₁₀ carbocyclyl group optionally substituted with one or more halogen, —OR^{a}, —SR^{a}, —NR^{a}R^{b}, —C(O)OR^{a}, —C(O)NR^{a}R^{b}, —C(O)R^{a} and/or C₁ to C₄ alkyl group(s) as described further below, or a heterocyclyl group selected from pyranyl, dihydropyranyl, dihydrofuranyl, dihydrobenzofuranyl, dihydroisobenzofuranyl, benzopyranyl, dihydrobenzopyranyl, furanyl and benzofuranyl, optionally substituted with one or more halogen, —OR^{a}, —SR^{a}, —NR^{a}R^{b}, —C(O)OR^{a}, —C(O)NR^{a}R^{b}, —C(O)R^{a} and/or C₁ to C₄ alkyl group(s) as described further below.

Preferably, in the compound of Formula (IA):

X represents O;

W represents an unsubstituted benzene or naphthalene ring;

Y represents O;

R¹ and R² each independently represent hydrogen; or

R¹ and R² are linked to form, together with W, a benzopyran or benzodihydropyran ring; and

n is 0 or 1;

preferably wherein Z is a bond and/or R³ represents a C₆ to C₁₀ aryl group optionally substituted by one or more hydroxyl, halogen and/or C₁ to C₄ alkyl groups.

In a further embodiment, the compound of the invention may be a compound of formula (II) or (III), wherein X, Z and R³ are as defined above.

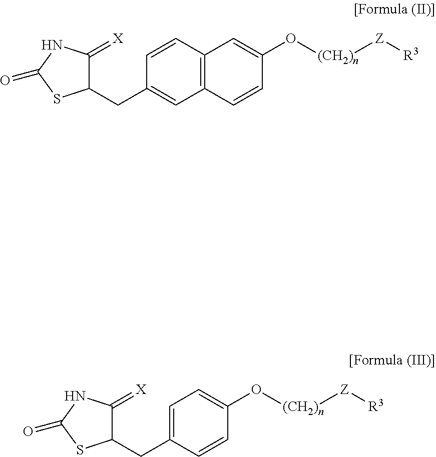

In the compound of Formula (II) or (III), n is an integer of 1 or 2, preferably 1.

Preferably in the compound of Formula (II) or (III), R³ represents a C₆ to C₁₀ aryl group optionally substituted with one or more halogen, —OR^{a}, —SR^{a}, —NR^{a}R^{b}, —C(O)OR^{a}, —C(O)NR^{a}R^{b}, —C(O)R^{a} and/or C₁ to C₄ alkyl group(s) as described further below, or a C₅ to C₁₀ carbocyclyl group optionally substituted with one or more halogen, —OR^{a}, —SR^{a}, —NR^{a}R^{b}, —C(O)OR^{a}, —C(O)NR^{a}R^{b}, —C(O)R^{a} or a C₁ to C₄ alkyl group(s) as described further below, or a heterocyclyl group selected from pyranyl, dihydropyranyl, dihydrofuranyl, dihydrobenzofuranyl, dihydroisobenzofuranyl, benzopyranyl, dihydrobenzopyranyl, furanyl and benzofuranyl, optionally substituted with one or more halogen, —OR^{a}, —SR^{a}, —NR^{a}R^{b}, —C(O)OR^{a}, —C(O)NR^{a}R^{b}, —C(O)R^{a} or a C₁ to C₄ alkyl group(s) as described further below.yl.

In one preferred embodiment of the compound of Formula (II) or (III):

X represents O;

n is an integer of 1 or 2;

Z is a bond; and

R³ represents a C₆ to C₁₀ aryl group optionally substituted by one or more hydroxyl, halogen and/or C₁ to C₄ alkyl groups.

In another embodiment, the compound of the invention is Netoglitazone, Ciglitazone, Englitazone, Darglitazone or Troglitazone. Preferably, the compound of the invention is Netoglitazone, Ciglitazone or Englitazone. In one embodiment, Netoglitazone is preferred in view of the fact that there is late-stage clinical data available for this compound.

As used herein, a C₆ to C₁₀ aryl group or moiety is an aryl group or moiety having from 6 to 10 carbon atoms, for example, phenyl or naphthyl, preferably phenyl. An aryl group or moiety can be substituted or unsubstituted. Suitable substituents include a halogen such as chlorine and/or fluorine, —OR^{a}, —SR^{a}, —NR^{a}R^{b}, —C(O)OR^{a}, —C(O)NR^{a}R^{b}, —C(O)R^{a} and a C₁ to C₄ alkyl group such as methyl and/or ethyl, wherein a C₁ to C₄ alkyl substituent is itself either unsubstituted or substituted with 1 to 3 halogen atoms. R^{a} and R^{b} are as defined herein.

As used herein, a C₅ to C₁₀ carbocyclyl group or moiety can be a C₅, C₆, C₇, C₈, C₉ or C₁₀ cycloalkyl group and is preferably cyclopentyl or cyclohexyl. Typically a cycloalkyl group is substituted or unsubstituted with up to three substituents, e.g. one or two substituents. Suitable substituents include a halogen such as chlorine and/or fluorine, —OR^{a}, —SR^{a}, —NR^{a}R^{b}, —C(O)OR^{a}, —C(O)NR^{a}R^{b}, —C(O)R^{a} and a C₁ to C₄ alkyl group such as methyl and/or ethyl, wherein a C₁ to C₄ alkyl substituent is itself either unsubstituted or substituted with 1 to 3 halogen atoms. R^{a} and R^{b} are as defined herein.

As used herein and unless otherwise stated, a 5- to 10-membered saturated heterocyclyl group or moiety is a saturated 5- to 10-membered ring system in which the ring contains at least one heteroatom. Typically, the ring contains up to three or four heteroatoms, e.g. one or two heteroatoms, selected from O, S and N. Thus, a 5- to 10-membered saturated heterocyclyl group or moiety is typically a 5- to 10-membered ring containing one, two or three heteroatoms selected from O, S and N. Suitable such heterocyclyl groups and moieties include, for example, monocyclic saturated 5- to 8-membered rings, more preferably 5- to 7-membered rings, such as tetrahydrofuranyl, piperidinyl, oxazolidinyl, morpholinyl, thiomorpholinyl, pyrrolidinyl, dioxolanyl, piperidonyl, azepanyl, oxepanyl, piperazinyl, tetrahydropyranyl and 1,4-diazepanyl, more preferably pyrrolidinyl, morpholinyl, piperazinyl, tetrahydropyranyl, piperidinyl, azepanyl and 1,4-diazepanyl.

As used herein and unless otherwise stated, a 5- to 10-membered unsaturated heterocyclic group or moiety is a 5- to 10-membered ring system in which the ring contains at least one unsaturated bond and at least one heteroatom. The ring may be partially unsaturated or fully unsaturated and aromatic. Typically, the ring contains up to three or four heteroatoms, e.g. one or two heteroatoms, selected from O, N and S. Thus, a 5- to 10-membered unsaturated heterocyclic group or moiety is typically a 5- to 10-membered ring containing one, two or three heteroatoms selected from O, N and S. Preferably, the heteroatoms are selected from O and N. Suitable such heterocyclyl groups and moieties include, for example:

monocyclic partially unsaturated 5- to 7-membered heterocyclyl rings such as dihydrofuranyl, pyranyl, dihydropyranyl, dioxinyl, dihydrooxepinyl, tetrahydrooxepinyl, pyrrolinyl, pyrazolinyl, imidazolinyl, dihydrooxazolyl, dihydroisoxazolyl, dihydrothiazolyl, dihydroisothiazolyl, dihydropyridinyl, tetrahydropyridinyl, dihydropyridazinyl, tetrahydropyridazinyl, dihydropyrimidinyl, tetrahydropyrimidinyl, dihydropyrazinyl, tetrahydropyrazinyl, oxazinyl, dihydrooxazinyl, thiazinyl, dihydrothiazinyl, dihydroazepinyl, tetrahydroazepinyl, dihydrothiophenyl, thiopyranyl, dihydrothiopyranyl, dihydrothiepinyl, and tetrahydrothiepinyl;

bicyclic partially unsaturated 8- to 10-membered heterocyclyl rings such as dihydrobenzofuranyl, dihydroisobenzofuranyl, benzopyranyl, dihydrobenzopyranyl, benzodioxolyl, indolinyl, isoindolinyl, dihydroquinolinyl, tetrahydroquinolinyl, benzooxazinyl, dihydrobenzothiophenyl and benzodithiole; preferably dihydrobenzofuranyl, benzopyranyl, dihydrobenzopyranyl, benzodioxolyl, indolinyl, isoindolinyl, dihydroquinolinyl and tetrahydroquinolinyl;

monocyclic 5- to 7-membered heteroaryl rings such as furanyl, oxepinyl, pyrrolyl, pyrazolyl, imidazolyl, triazolyl, tetrazolyl, oxazolyl, isoxazolyl, oxadiazolyl, thiazolyl, isothiazolyl, thiadiazolyl, pyridinyl, pyradazinyl, pyrimidinyl, pyrazinyl, triazinyl, azepinyl, thiophenyl, oxepinyl and thiepinyl; and

bicyclic 8- to 10-membered heteroaryl rings such as benzofuranyl, indolyl, isoindolyl, indolizinyl, indazolyl, benzimidazolyl, azaindolyl, azaindazolyl, purinyl, benzooxazolyl, benzoisooxazolyl, benzothiazolyl, benzoisothiazolyl, benzothiadiazolyl, quinolinyl, isoquinolinyl, quinolizinyl, quinoxalinyl, phthalazinyl, quinazolinyl, cinnolinyl, naphthyridinyl, pteridinyl and benzothiophenyl, preferably benzofuranyl, indolyl, isoindolyl, quinolinyl and isoquinolinyl.

Preferably, the 5- to 10-membered unsaturated heterocyclic group is a monocyclic partially unsaturated 5- to 7-membered ring selected from dihydrofuranyl, pyranyl, pyrrolinyl and oxazinyl or a monocyclic 5- to 7-membered heteroaryl ring selected from furanyl, pyrrolyl, pyrazolyl, imidazolyl, triazolyl, oxazolyl, isoxazolyl, pyridinyl, pyradazinyl, pyrimidinyl and pyrazinyl.

As used herein and unless otherwise stated, a 5- to 10-membered partially unsaturated heterocyclyl group or moiety which does not contain a nitrogen heteroatom in the ring is a 5-to 10-membered ring system in which the ring contains at least one unsaturated bond and at least one heteroatom and does not contain a nitrogen heteroatom. Typically, the ring contains up to three or four heteroatoms, e.g. one or two heteroatoms, selected from O and S. Thus, a 5- to 10-membered partially unsaturated heterocyclyl group or moiety is typically a 5- to 10-membered ring containing one, two or three heteroatoms selected from O and S. Preferably, the heteroatom(s) are O. Suitable such heterocyclyl groups and moieties include, for example, monocyclic partially unsaturated 5- to 7-membered heterocyclyl rings such as pyranyl, thiopyranyl, dihydropyranyl, dihydrothiopyranyl, dioxinyl, dihydrofuranyl, dihydrothiophenyl, dihydrooxepinyl, dihydrothiepinyl, tetrahydrooxepinyl, tetrahydrothiepinyl, preferably pyranyl, thiopyranyl, dihydropyranyl and dihydrofuranyl; and bicyclic partially unsaturated 8- to 10-membered heterocyclyl rings such as dihydrobenzofuranyl, dihydroisobenzofuranyl, benzopyranyl, dihydrobenzopyranyl, benzodioxolyl, dihydrobenzothiophenyl, and benzodithiole. Preferably, the 5- to 10-membered partially unsaturated heterocyclyl group is selected from pyranyl, dihydropyranyl, dihydrofuranyl, dihydrobenzofuranyl, dihydroisobenzofuranyl, benzopyranyl and dihydrobenzopyranyl.

As used herein, and unless otherwise stated, a 5- to 10-membered heteroaryl group or moiety which does not contain a nitrogen heteroatom in the ring is a 5- to 10-membered ring system in which the ring is fully unsaturated and aromatic, contains at least one heteroatom and does not contain a nitrogen heteroatom. Typically, the ring contains up to three or four heteroatoms, e.g. one or two heteroatoms, selected from O and S. Thus, a 5- to 10-membered heteroaryl group or moiety is typically a 5- to 10-membered ring containing one, two or three heteroatoms selected from O and S. Preferably, the heteroatom(s) are O. Suitable such heteroaryl groups and moieties include, for example, monocyclic 5- to 7-membered heteroaryl rings, such as furanyl, thiophenyl, oxepinyl and thiepinyl; and bicyclic 8- to 10-membered heteroaryl rings such as benzofuranyl and benzothiophenyl. Preferably, the 5- to 10-membered hetereoaryl group is selected from furanyl and benzofuranyl.

A heterocyclyl and/or heteroaryl group or moiety may be substituted or unsubstituted. Each ring atom may be unsubstituted or may carry one or two substituents. If desired, a nitrogen atom may be disubstituted and a sulphur atom may be substituted, providing a charged heteroatom. Typically, a heterocyclyl or aryl group or moiety carries up to three substituents, e.g. one or two substituents. The heterocycle may be connected to the remainder of the molecule by a bond to any of its available ring positions.

As used herein, a group which is optionally substituted may be substituted with suitable substituents which include a halogen such as chlorine and/or fluorine, —OR^{a}, —SR^{a}, —NR^{a}R^{b}, —C(O)OR^{a}, —C(O)NR^{a}R^{b}, —C(O)R^{a} and a C₁ to C₄ alkyl group such as methyl and/or ethyl, wherein a C₁ to C₄ alkyl substituent is itself either unsubstituted or substituted with 1 to 3 halogen atoms. R^{a} and R^{b} are as defined below. The optional substituent is preferably a hydroxyl, halogen such as chlorine or fluorine, or C₁ to C₄ alkyl group such as methyl or ethyl.

As used herein, a halogen is typically chlorine, fluorine, bromine or iodine, and is preferably chlorine, fluorine or bromine, more preferably chlorine or fluorine.

A C₁ to C₄ alkyl group or moiety can be linear, branched or cyclic but is preferably linear. Suitable such alkyl groups and moieties include methyl, ethyl, n-propyl, i-propyl, n-butyl, sec-butyl and tert-butyl. It is preferably a C₁ to C₃ alkyl group, more preferably ethyl or methyl. An alkyl group or moiety can be unsubstituted or substituted with 1, 2 or 3 halogen atoms.

As used herein, each R^{a} and each R^{b} independently represents hydrogen or an unsubstituted C₁ to C₄ alkyl group.

The compounds of the present invention may be produced using known methods. In particular, Netoglitazone is a known compound and can be produced, for example, according to the methods described in JP2009/234930 and WO2000/31055 or methods complying therewith.

The compound of the invention containing one or more chiral centre(s) may be used in enantiomerically or diastereomerically pure form or in the form of a mixture of isomers. The compounds of the invention may be used in any tautomeric form.

The compound can be used in the form of a pharmaceutically acceptable salt. As used herein, a pharmaceutically acceptable salt is a salt with a pharmaceutically acceptable acid or base. Pharmaceutically acceptable acids include both inorganic acids such as hydrochloric, sulphuric, phosphoric, diphosphoric, hydrobromic, hydroiodic or nitric acid and organic acids such as citric, fumaric, maleic, malic, ascorbic, succinic, tartaric, benzoic, acetic, methanesulphonic, ethanesulphonic, benzenesulphonic, p-toluenesulphonic acid, formic, acetic, propionic, glycolic, lactic, pyruvic, oxalic, salicylic, trichloroacetic, picric, trifluoroacetic, cinnamic, pamoic, malonic, mandelic, bismethylene salicylic, ethanedisulfonic, gluconic, citraconic, aspartic, stearic, palmitic, EDTA, p-aminobenzoic or glutamic acid, sulfates, nitrates, phosphates, perchlorates, borates, acetates, benzoates, hydroxynaphthoates, glycerophosphates or ketoglutarates. Further examples of pharmaceutically acceptable inorganic or organic acid addition salts include the pharmaceutically acceptable salts listed in Journal of Pharmaceutical Science, 66, 2 (1977) which are known to the skilled artisan. Pharmaceutically acceptable bases include alkali metal (e.g. sodium or potassium) and alkali earth metal (e.g. calcium or magnesium) hydroxides and organic bases such as alkyl amines, aralkyl amines and heterocyclic amines, lysine, guanidine, diethanolamine and choline.

The acid addition salts may be obtained as the direct products of compound synthesis. In the alternative, the free base may be dissolved in a suitable solvent containing the appropriate acid, and the salt isolated by evaporating the solvent or otherwise separating the salt and the solvent.

The compound of the invention may be used in the form of a solvate or hydrate. The compound may form solvates with standard low molecular weight solvents using methods known to the skilled artisan.

The present invention also provides prodrugs of the compounds of the invention. A prodrug is an analogue of a compound of the invention which will be converted in vivo to the desired active compound. Examples of suitable prodrugs include compounds which have been modified at a carboxylic acid group to form an ester, or at hydroxyl group to form an ester or carbamate. Further suitable prodrugs include those in which a nitrogen atom of the compound is quaternised by addition of an ester or alkyl ester group. For example, the nitrogen atom of an amine group or heterocyclyl ring may be quaternised by the addition of a —CH₂—O—COR group, wherein R is typically methyl or tert-butyl. Other suitable methods will be known to those skilled in the art.

The present invention further provides precursors of the compounds of the invention. A precursor is a compound which the person skilled in the art could trivially convert into the desired active compound. Examples of suitable precursors include compounds which can be converted into compounds of the invention by the removal of a protecting group by a process known in the art.

The present invention also provides isotopically labelled derivatives of the compounds of the invention (or pharmaceutically acceptable salts, tautomers, solvates, hydrates, prodrugs, derivatives, stereoisomers or analogs thereof). An isotopically labelled derivative is a compound in which one or more of the constituent atoms are an atom having an atomic mass or mass number different from the atomic mass or mass number most commonly found in nature. Examples of isotopes suitable for inclusion in the compound of the invention include isotopes of: hydrogen, such as ²H and ³H; carbon, such as ¹¹C, ¹³C and ¹⁴C; nitrogen, such as ¹³N, ¹⁵N and ¹⁶N; oxygen, such as ¹⁵O, ¹⁷O and ¹⁸O; fluorine, such as ¹⁸F; phosphorous, such as ³²P; sulphur, such as ³⁵S; chlorine, such as ³⁶C₁; bromine, such as ⁷⁷Br; and iodine, such as ¹²³I and ¹²⁵I. Preferred isotopes are ²H, ³H, ¹³C, ¹⁵N, ¹⁸O, ¹⁸F, ³⁶Cl, and ⁷⁷Br.

Substitution with heavier isotopes such as deuterium, ²H, may afford certain therapeutic advantages resulting from greater metabolic stability, such as increased in vivo half-life or reduced dosage requirements. Such isotopically-labelled compounds of the invention may therefore be preferable in some circumstances.

Isotopically labelled compounds of the invention can be prepared by conventional techniques known to those skilled in the art, for example by carrying out isotopic substitution reactions or by using isotopically labelled reagents in place of non-labelled reagents.

Preferably, the compound for use according to the present invention is a compound of the invention or a pharmaceutically acceptable salt, tautomer, solvate, hydrate, prodrug, stereoisomer or isotopically labelled derivative thereof. More preferably, the compound for use is a compound of the invention or a pharmaceutically acceptable salt, tautomer, solvate, hydrate, stereoisomer or isotopically labelled derivative thereof.

Treatment

In protein misfolding disease, it is typical for the misfolded protein to display an increased tendency to bind to itself and thus form protein oligomers, aggregates and fibrils. This is often associated with an increase in the formation of a β-sheet secondary protein structure. These aggregates are resistant to the normal cellular clearance of proteins and therefore accumulate, potentially forming plaques consisting of large aggregates. This can cause cell death and/or abnormal function of the affected tissue. The formation and growth of these aggregates involves the generation of new aggregates and the propagation of existing aggregates. Thus, protein misfolding diseases are commonly caused, symptomised by or otherwise associated with the formation, accumulation, deposition and persistence of such oligomers, aggregates, fibrils and/or plaques of proteins and/or peptides. A treatment for protein misfolding diseases such as that provided by the present invention may therefore target such aggregated species.

Thus, in one embodiment the compound of the invention may be for use in treating, preventing or inhibiting the formation, deposition, accumulation or persistence of oligomers, fibrils, aggregates and/or plaques of proteins and/or peptides.

Amyloidogenic proteins are an example of proteins with a tendency to aggregate, and these proteins can misfold and aggregate leading to amyloidosis diseases. The amyloid precursor protein can undergo proteolysis to generate the Aβ peptide whose fibrillary form is associated with various protein misfolding diseases, particularly AD.

In a preferred embodiment, the compound of the invention is for use in treating, preventing or inhibiting the formation, deposition, accumulation or persistence of amyloid oligomers, fibrils, aggregates and/or plaques. More preferably, the amyloid oligomers, fibrils, aggregates and/or plaques are amyloid-β oligomers, fibrils, aggregates and/or plaques.

Protein aggregation in the brain is a very complex and multi-factorial process and it has proved very difficult to obtain accurate knowledge regarding the molecular mechanisms underlying the generation of toxic species and the process by which small molecules interfere with the aggregation pathway. Widespread evidence suggests that pre-fibrillar oligomeric species, rather than mature amyloid plaques, are the primary pathogenic agents. These oligomeric species are challenging to characterise due to their transient nature, which complicates drug discovery. This amongst many other evidences suggest that effective therapeutic strategies are unlikely to consist of a nonspecific suppression of the fibril formation process, such as the ones that were widely used to identify drugs and that have systematically failed clinical trials, but rather to involve the targeting of specific species in a controlled intervention at a precise microscopic step during the greatly complex and heterogeneous aggregation process.

Recent advances in establishing rate laws in chemical kinetics have allowed the details of Aβ macroscopic kinetic measurements to be finely described at the microscopic levels. The establishment of rate laws allowed at least three different classes of microscopic processes to be distinguished. The generation of aggregates can occur through either primary pathways, where new aggregates form from soluble monomers, or through secondary pathways. In the secondary pathways, new aggregates proliferate though either fragmentation, which is monomer-independent, or through surface catalysed secondary nucleation, which is monomer dependent.

As a consequence of this development, a key discovery has been made showing that the dominant mechanism responsible for the generation of toxic Aβ species in AD is a specific step in the aggregation process, namely the surface-catalysed secondary nucleation. This finding is clearly important because unlike previous non-specific inhibition of aggregation measurements, it allows for the toxic process to be specifically targeted. This advance has also led to the conclusion that inhibiting Aβ aggregation per se, without an accurate knowledge of the underlying microscopic processes, could have unexpected consequences on the toxicity. Indeed, it could not only decrease it, but also leave it unaffected, or even increase it in the case the wrong microscopic step is targeted. Furthermore, the application of chemical kinetics does not require prior knowledge of the structure of the pathogenic species and it is not limited by the need for high protein-molecule binding affinities. Accordingly, the identification of efficient inhibitors that can perturb a specific microscopic step in Aβ42 aggregation could provide an efficient strategy for suppressing pathogenicity.

In one embodiment, the treating, preventing or inhibiting the formation, deposition, accumulation, or persistence of protein and/or peptide oligomers, fibrils, aggregates and/or plaques as discussed above may be achieved by inhibiting the primary nucleation and/or the surface-catalysed secondary nucleation of such oligomers, fibrils, aggregates and/or plaques. Preferably, this is achieved by inhibiting both the primary nucleation and secondary nucleation of oligomers, fibrils, aggregates and/or plaques. The oligomers, fibrils, aggregates and/or plaques are preferably Aβ oligomers, fibrils, aggregates and/or plaques, as discussed above.

The compounds of the invention may have anti-proteinopathic properties. Accordingly, they may be used in a method of treating a subject suffering from or susceptible to a protein misfolding disease, which method comprises administering to said subject an effective amount of the compound of the invention or a pharmaceutically acceptable salt, tautomer, solvate, hydrate, prodrug, derivative, stereoisomer, analog or isotopically labelled derivative thereof. The compounds may be used in combination with additional therapeutic agent(s), as desired.

Multiple proteinopathies can overlap and multiple proteins can be associated with a protein misfolding disease. For example, Parkinson's disease is primarily associated with the misfolding of α-synuclein peptides but is additionally associated with the misfolding of Aβ peptides. Given the general phenomenon of protein aggregation, drugs which are known to be effective in the treatment and/or prevention of the misfolding of one peptide may be modified to be effective in the treatment and/or prevention of the misfolding of other peptides.

In the present invention, the protein misfolding disease is preferably associated with misfolding of the Aβ peptide. Thus, for example, the Aβ peptide may cause, symptomize and/or otherwise be associated with the protein misfolding disease. In some embodiments, the protein misfolding disease is one or more disease selected from: amyloidosis (including Alzheimer's disease, AL and AA amyloidosis), primary and secondary tauopathies (including Alzheimer's disease, Progressive supranuclear palsy and Primary age-related tauopathy), prion diseases (including Creutzfeld-Jakob disease, spongiform encephalopathies, kuru), neurodegenerative disease (including Alzheimer's disease, Parkinson's disease, dementia with Lewy Bodies), Down syndrome and/or cystic fibrosis.

In one embodiment, the protein misfolding disease may be a neurodegenerative disease. The neurodegenerative disease may be, for example, dementia (including Alzheimer's disease), mild cognitive impairment (MCI), Parkinson's disease, polyglutamine diseases (such as Huntington's disease) and/or amyotrophic lateral sclerosis (ALS). Preferably, the neurodegenerative disease is dementia. More preferably, the dementia is selected from Alzheimer's disease, dementia with Lewy Bodies, frontotemporal dementia, familial dementia and/or progressive supranuclear palsy (PSP). Preferably, the dementia is Alzheimer's disease.

As noted above, the disease may be predominantly caused by, symptomised by, or otherwise associated with misfolding of the amyloid-β peptide. Thus the disease may be Alzheimer's disease, cerebral amyloid-β angiopathy, inclusion body myositis and/or Down syndrome, preferably Alzheimer's disease.

In one embodiment, the compound of the invention or a pharmaceutically acceptable salt, tautomer, solvate, hydrate, prodrug, derivative, stereoisomer, analog or isotopically labelled derivative thereof as discussed herein is for use in the treatment and/or prevention of a neurodegenerative disease, for example dementia (such as Alzheimer's disease, dementia with Lewy Bodies, frontotemporal dementia, familial dementia and/or progressive supranuclear palsy (PSP)), mild cognitive impairment (MCI), Parkinson's disease, polyglutamine diseases (such as Huntington's disease) and/or amyotrophic lateral sclerosis (ALS). Preferably, the dementia is Alzheimer's disease.

In a preferred embodiment, the compound is for use in the treatment of a patient which has been diagnosed with Alzheimer's disease.

The Reisberg scale, also known as the Global Deterioration Scale, is a system commonly used by healthcare professionals and caregivers to classify the severity and degenerative progression of an incidence of neurodegenerative dementia such as Alzheimer's disease. The seven stages on the scale are defined by typical symptomatic losses of cognitive function. Stage 1 is pre-symptomatic. Stages 2 and 3 classify mild Alzheimer's disease and are often considered to be ‘pre-dementia’, as the cognitive decline is evident but does not significantly impact the patient's life. Stage 2 and 3 Alzheimer's disease can be classified as Mild Cognitive Impairment (MCI), also known as incipient dementia. Stages 4 to 7 are classified as dementia. From stage 5 onwards, a patient is considered to require living assistance.

In the present invention wherein the compound of the invention or a pharmaceutically acceptable salt, tautomer, solvate, hydrate, prodrug, derivative, stereoisomer, analog or isotopically labelled derivative thereof is for use in preventing or treating Alzheimer's disease, the Alzheimer's disease is preferably stage 1 to stage 4 Alzheimer's disease. More preferably, it is stage 1 to 3 Alzheimer's disease, such as stage 2 or stage 3 Alzheimer's disease.

MCI is a neurological disorder symptomised by an onset and progression of cognitive impairment beyond that expected based on the age and education of the individual, but which does not significantly disrupt daily activities. When the predominant symptom is memory loss, the disorder is termed “amnestic MCI” and is widely considered to be a prodromal stage of Alzheimer's disease. Patients with amnestic MCI develop Alzheimer's disease at a rate of approximately 10 to 15% per year.

In one embodiment of the present invention, the compound is for use in the treatment of a patient which is at risk of developing Alzheimer's disease. Preferably, the patient has been diagnosed with MCI. Furthermore, the patient preferably has a family history of Alzheimer's disease. When the patient is at risk of developing Alzheimer's disease, has been diagnosed with MCI, and/or has a family history of Alzheimer's disease, early stage intervention is possible and the formation of plaques can be avoided or reduced. This presents an opportunity for developing an effective strategy for preventing or delaying the onset of symptoms. In particular, the compound of the present invention is highly effective at preventing the nucleation of Aβ aggregates and may therefore be particularly effective when used as an early stage intervention.

Studies of protein misfolding in Alzheimer's disease suggest that the earlier stages of Alzheimer's disease are primarily associated with the Aβ peptide and the formation of extracellular amyloid plaques, while later stages are symptomised by the misfolding of tau peptides into intraneuronal neurofibrillary tangles. A prevailing theory is that the upstream Aβ misfolding plays a role in triggering the conversion of tau from a normal to a toxic state. There is evidence that the toxic tau species enhance the misfolded Aβ toxicity and vice versa in a toxic feedback loop, enhancing neurodegeneration. A therapeutic strategy for Alzheimer's disease which targeted the Aβ peptide and prevented this triggering of tau toxicity by preventing or reducing the severity of Aβ misfolding would therefore provide a powerful treatment in the prevention and/or delay of the onset of symptoms and late stage AD, and particularly the more severe symptoms of AD.

The present invention additionally provides a method of treating and/or preventing a protein misfolding disease and/or a neurodegenerative disease as described above in a patient which comprises administering to said patient an effective amount of a compound of the present invention as described above or a pharmaceutically acceptable salt, tautomer, solvate, hydrate, prodrug, derivative, analog or isotopically labelled derivative thereof. Preferred features of the compound for use as defined herein are also preferred features of the method of the invention.

The present invention further provides the use of a compound of the present invention as described above or a pharmaceutically acceptable salt, tautomer, solvate, hydrate, prodrug, derivative, analog or isotopically labelled derivative thereof in the manufacture of a medicament for the treatment and/or prevention of a protein misfolding disease and/or a neurodegenerative disease as described above. Preferred features of the compound for use as defined herein are also preferred features of the use of the invention.

In one preferred embodiment, the present invention relates to a compound of Formula (I) as discussed above, or a pharmaceutically acceptable salt, tautomer, solvate, hydrate, prodrug, derivative, stereoisomer, analog or isotopically labelled derivative thereof, for use in the treatment and/or prevention of a protein misfolding disease selected from amyloidosis, tauopathies, prion diseases (including Creutzfeld-Jakob disease and spongiform encephalopathies), neurodegenerative disease, Down syndrome, and/or cystic fibrosis as discussed above; a protein misfolding disease selected from Alzheimer's disease, cerebral amyloid-β angiopathy, inclusion body myositis and/or Down's syndrome as discussed above; and/or a neurodegenerative disease selected from dementia, mild cognitive impairment (MCI), Parkinson's disease, polyglutamine diseases (such as Huntington's disease) and/or amyotrophic lateral sclerosis (ALS) as discussed above. Preferably, the compound is for use in the treatment of Alzheimer's disease.

In another preferred embodiment, the present invention relates to a compound of Formula (IA) as discussed above, or a pharmaceutically acceptable salt, tautomer, solvate, hydrate, prodrug, derivative, stereoisomer, analog or isotopically labelled derivative thereof, for use in the treatment and/or prevention of a protein misfolding disease selected from amyloidosis, tauopathies, prion diseases (including Creutzfeld-Jakob disease and spongiform encephalopathies), neurodegenerative disease, Down syndrome, and/or cystic fibrosis as discussed above; a protein misfolding disease selected from Alzheimer's disease, cerebral amyloid-β angiopathy, inclusion body myositis and/or Down's syndrome as discussed above; and/or a neurodegenerative disease selected from dementia, mild cognitive impairment (MCI), Parkinson's disease, polyglutamine diseases (such as Huntington's disease) and/or amyotrophic lateral sclerosis (ALS) as discussed above. Preferably, the compound is for use in the treatment of Alzheimer's disease.

In another preferred embodiment, the present invention relates to a compound of Formula (II) or (III) as discussed above, or a pharmaceutically acceptable salt, tautomer, solvate, hydrate, prodrug, derivative, stereoisomer, analog or isotopically labelled derivative thereof, for use in the treatment and/or prevention of a protein misfolding disease selected from amyloidosis, tauopathies, prion diseases (including Creutzfeld-Jakob disease and spongiform encephalopathies), neurodegenerative disease, Down syndrome, and/or cystic fibrosis as discussed above; a protein misfolding disease selected from Alzheimer's disease, cerebral amyloid-β angiopathy, inclusion body myositis and/or Down's syndrome as discussed above; and/or a neurodegenerative disease selected from dementia, mild cognitive impairment (MCI), Parkinson's disease, polyglutamine diseases (such as Huntington's disease) and/or amyotrophic lateral sclerosis (ALS) as discussed above. Preferably, the compound is for use in the treatment of Alzheimer's disease.

In another preferred embodiment, the present invention relates to Netoglitazone, Ciglitazone, Englitazone, Darglitazone or Troglitazone, preferably Netoglitazone, Ciglitazone, or Englitazone, more preferably Netoglitazone, or a pharmaceutically acceptable salt, tautomer, solvate, hydrate, prodrug, derivative, stereoisomer, analog or isotopically labelled derivative thereof, for use in the treatment and/or prevention of a protein misfolding disease selected from amyloidosis, tauopathies, prion diseases (including Creutzfeld-Jakob disease and spongiform encephalopathies), neurodegenerative disease, Down syndrome, and/or cystic fibrosis as discussed above; a protein misfolding disease selected from Alzheimer's disease, cerebral amyloid-β angiopathy, inclusion body myositis and/or Down's syndrome as discussed above; and/or a neurodegenerative disease selected from dementia, mild cognitive impairment (MCI), Parkinson's disease, polyglutamine diseases (such as Huntington's disease) and/or amyotrophic lateral sclerosis (ALS) as discussed above. Preferably, the said compound is for use in the treatment of Alzheimer's disease.

Pharmaceutical Compositions and Administration

The present invention also provides a pharmaceutical composition comprising the compound of the invention or a pharmaceutically acceptable salt, tautomer, solvate, hydrate, prodrug, derivative, stereoisomer, analog or isotopically labelled derivative thereof for use in treating and/or preventing protein misfolding disease. In one embodiment, this composition further comprises one or more pharmaceutically acceptable carriers diluents, excipients and/or additives. Preferred features of the compound for use as defined herein are also preferred features of the composition for use.

Preferably, the composition is a solution of the compound of the invention in a liquid carrier. Preferred pharmaceutical compositions are sterile.

The concentration of the compound of the invention in a pharmaceutical composition will vary depending on several factors, including the dosage of the compound to be administered.

In one embodiment, the compound of the invention is administered as a monotherapy. In another embodiment, the present invention provides a pharmaceutical combination of the compound of the invention or a pharmaceutically acceptable salt, tautomer, solvate, hydrate, prodrug, derivative, stereoisomer, analog or isotopically labelled derivative thereof, with one or more additional therapeutic agent(s), wherein the additional therapeutic agent(s) are suitable for the treatment and/or prevention of protein misfolding disease. Thus, the compound of the invention is present in the combinations, compositions and products of the invention with one or more additional therapeutic agent(s).

In one embodiment the present invention provides a pharmaceutical composition comprising (i) a compound of the invention or a pharmaceutically acceptable salt, tautomer, solvate, hydrate, prodrug, derivative, stereoisomer, analog or isotopically labelled derivative thereof, (ii) one or more additional therapeutic agent(s), which additional therapeutic agent(s) may be as defined herein and (iii) one or more pharmaceutically acceptable carriers and/or excipients.

Typically, the combination is a combination in which the compound of the invention or a pharmaceutically acceptable salt, tautomer, solvate, hydrate, prodrug, derivative, stereoisomer, analog or isotopically labelled derivative thereof, and the additional therapeutic agent(s) are formulated for separate, simultaneous or successive administration. The combination may optionally also comprise a pharmaceutically acceptable carrier or diluent.

When, for example, the compound of the invention is part of a combination (such as a pharmaceutical combination) as defined herein, formulated for separate, simultaneous or successive administration, (a) the pharmaceutical compound of the invention, and (b) the additional therapeutic agent(s) may be administered by the same mode of administration or by different modes of administration.

For simultaneous administration, the compound of the invention or a pharmaceutically acceptable salt, tautomer, solvate, hydrate, prodrug, derivative, stereoisomer, analog or isotopically labelled derivative thereof, and the additional therapeutic agent(s) may for example be provided in a single composition. Thus, the composition may, for example, comprise the compound of the invention or a pharmaceutically acceptable salt, tautomer, solvate, hydrate, prodrug, derivative, stereoisomer, analog or isotopically labelled derivative thereof, and the additional therapeutic agent(s), and optionally a pharmaceutically acceptable carrier or diluent. For separate or successive administration, the compound of the invention or a pharmaceutically acceptable salt, tautomer, solvate, hydrate, prodrug, derivative, stereoisomer, analog or isotopically labelled derivative thereof, and the additional therapeutic agent(s) may, for example, be provided as a kit.

The additional therapeutic agent(s) used in the invention can be any suitable therapeutic agent that the skilled person would judge to be useful in the circumstances. Particularly suitable classes of therapeutic agents include drugs targeting the following pathways or mechanisms: acetylcholine (e.g. Acetylcholine agonists, Acetylcholinesterase inhibitors, Nerve Growth Factor enhancers), inflammation (e.g. Lipoprotein-associated phospholipase A2 inhibitors, Phosphodiesterase (PDE) inhibitor), serotonin (e.g. 5-HTR antagonists, Monoamine oxidases inhibitors), glutamate (e.g. NMDA antagonist), antioxidants (GABA modulators, Dopamine, Cannabinoids), histamine, Aβ (e.g. aggregation inhibitors, passive immunotherapies, BACE inhibitors, γ-secretase modulators, PKC activators, other APP related enzymes), tau (e.g. aggregation inhibitors, passive immunotherapies, prevention of tau phosphorylation), immune therapies (e.g. vaccines against full-length or fragments of Aβ or tau with or without adjuvants), Insulin (PPAR and GLP-1), 11β-hydroxysteroid dehydrogenase 1 inhibitors, Mesenchymal stem cells transplant, Antisense oligonucleotide that inhibits MAPT, AAV to deliver MAPT antibodies. In a preferable embodiment, the additional therapeutic agent(s) are suitable for the treatment and/or prevention of a protein misfolding disease and/or a neurodegenerative disease. Preferably, the composition of the present invention is formulated to improve the penetration of the compound of the invention into the brain of the patient. This could be achieved, for example, through use of solid, colloidal particles (size 10-1000 nm) as drug carriers (e.g. Lipid-based nanoparticles: Solid lipid nanoparticles, Liposomes, Micelles, Nanoemulsions; Polymer-based nanoparticles: Dendrimers and Polymeric nanoparticles; Inorganic nanoparticles). A further approach is intranasal administration, a non-invasive drug delivery technique that bypasses the blood-brain barrier (BBB) via the olfactory nerves where the drug is directly delivered from the nasal mucosa to the brain by transcellular absorption or endocytosis. Other approaches include receptor based delivery systems (e.g. Transferrin TfR); chemical BBB modulatords (e.g. Borneol); ultrasound BBB disruption, and protein capsules.

The compound, combinations, compositions and products of the invention may be administered in a variety of dosage forms. Thus, they can be administered orally, for example as tablets, troches, lozenges, aqueous or oily suspensions, dispersible powders or granules. The compound, combinations, compositions and products of the invention may also be administered parenterally, either subcutaneously, intravenously, intramuscularly, intrasternally, transdermally or by infusion techniques. Depending on the vehicle and concentration used, the drugs can either be suspended or dissolved in the vehicle. Advantageously, adjuvants such as a local anaesthetic, preservative and buffering agent can be dissolved in the vehicle. The compound, combinations, compositions and products may also be administered as suppositories. The compounds, combinations, compositions and products may be administered by inhalation in the form of an aerosol via an inhaler or nebuliser. The pharmaceutical compound of the invention, pharmaceutical combinations and pharmaceutical compositions may be administered topically, for example, as a cream, foam, gel, lotion, or ointment.

A compound of the invention, and optionally additional therapeutic agent(s), is typically formulated for administration with a pharmaceutically acceptable carrier or diluent. For example, solid oral forms may contain, together with the active compound, solubilising agents, e.g. cyclodextrins or modified cyclodextrins; diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, arabic gums, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non-toxic and pharmacologically inactive substances used in pharmaceutical formulations. Such pharmaceutical preparations may be manufactured in known manner, for example, by means of mixing, granulating, tabletting, sugar-coating, or film coating processes.

Liquid dispersions for oral administration may be solutions, syrups, emulsions and suspensions. The solutions may contain solubilising agents e.g. cyclodextrins or modified cyclodextrins. The syrups may contain as carriers, for example, saccharose or saccharose with glycerine and/or mannitol and/or sorbitol.

Suspensions and emulsions may include pharmaceutically active compounds in which the average particle size has undergone particle size reduction by micronisation or nanonisation technologies. For instance, the average particle size of the compound of the invention may have undergone particle size reduction by micronisation or nanonisation technologies.

Suspensions and emulsions may contain as carrier, for example a natural gum, agar, sodium alginate, pectin, methylcellulose, carboxymethylcellulose, or polyvinyl alcohol. The suspensions or solutions for intramuscular injections may contain, together with the active compound, a pharmaceutically acceptable carrier, e.g. sterile water, olive oil, ethyl oleate, glycols, e.g. propylene glycol; solubilising agents, e.g. cyclodextrins or modified cyclodextrins, and if desired, a suitable amount of lidocaine hydrochloride.

Solutions for intravenous or infusions may contain as carrier, for example, sterile water and solubilising agents, e.g. cyclodextrins or modified cyclodextrins or preferably they may be in the form of sterile, aqueous, isotonic saline solutions.

For topical application to the skin, the compound may, for example, be made up into a cream, lotion or ointment. Cream or ointment formulations which may be used for the drug are conventional formulations well known in the art, for example as described in standard textbooks of pharmaceutics such as the British Pharmacopoeia.

For topical application by inhalation, the compound may be formulated for aerosol delivery for example, by pressure-driven jet atomizers or ultrasonic atomizers, or preferably by propellant-driven metered aerosols or propellant-free administration of micronized powders, for example, inhalation capsules or other “dry powder” delivery systems. Excipients, such as, for example, propellants (e.g. Frigen in the case of metered aerosols), surface-active substances, emulsifiers, stabilizers, preservatives, flavorings, and fillers (e.g. lactose in the case of powder inhalers) may be present in such inhaled formulations. For the purposes of inhalation, a large number of apparata are available with which aerosols of optimum particle size can be generated and administered, using an inhalation technique which is appropriate for the patient. In addition to the use of adaptors (spacers, expanders) and pear-shaped containers (e.g. Nebulator®, Volumatic®), and automatic devices emitting a puffer spray (Autohaler®), for metered aerosols, in particular in the case of powder inhalers, a number of technical solutions are available (e.g. Diskhaler®, Rotadisk®, Turbohaler® or the inhalers for example as described in European Patent Application EP 0 505 321).

A therapeutically effective amount of the compound of the invention or a pharmaceutically acceptable salt, tautomer, solvate, hydrate, prodrug, derivative, stereoisomer, analog or isotopically labelled derivative thereof is administered to a patient. A typical daily dose is, for example, from 0.1 to 25, from 0.2 to 20 or from 0.5 to 15 mg per kg of body weight, according to the activity of the compound or combination of specific therapeutic agents used, the age, weight and conditions of the subject to be treated, the type and severity of the disease and the frequency and route of administration. In one embodiment the daily dosage level is from 10 to 1500 mg, preferably from 15 to 1000 mg, and more preferably from 20 to 500 mg. Where a combination is administered, the compound of the invention or a pharmaceutically acceptable salt, tautomer, solvate, hydrate, prodrug, derivative, stereoisomer, analog or isotopically labelled derivative thereof is typically administered in an amount of at least 1 mg, preferably at least 5 mg, 10 mg or at least 20 mg. A preferred upper limit on the amount of compound of the invention or a pharmaceutically acceptable salt, tautomer, solvate, hydrate, prodrug, derivative, stereoisomer, analog or isotopically labelled derivative thereof administered is typically 200 mg, e.g. 100 mg, 50 mg or 25 mg. The compound of the invention or a pharmaceutically acceptable salt, tautomer, solvate, hydrate, prodrug, derivative, stereoisomer, analog or isotopically labelled derivative thereof is typically administered in twice daily dosages of 5 to 50 mg, preferably 10 to 40 mg and more preferably 15 to 30 mg. Any additional therapeutic agent(s) are typically administered at or below the standard dose used for that drug. The compound, combination or composition of the invention is typically administered to the patient in a non-toxic amount.

In an embodiment of the present invention, the compound or composition of the invention is administered such that the compound of the invention is administered in a daily dose of from 0.1 mg/kg to 25 mg/kg. Preferably, the compound of the invention is administered in a daily dose of from 0.5 mg/kg to 15 mg/kg.

In another embodiment, the compound is administered in a daily dose of from 10 mg to 1500 mg. Preferably, the compound is administered in a daily dose of from 20 mg to 500 mg.

In a further embodiment, the compound may be administered in a twice daily dose of from 5 mg to 50 mg, preferably in a twice daily dose of from 15 mg to 25 mg.

In an embodiment of the invention, the compound or composition of the invention is delivered in vivo in a mammal. In another embodiment the mammal is a human. In another specific embodiment the human has been diagnosed with AD, is known to have AD, is suspected of having AD, or is at risk for developing AD. In another embodiment, the human is known to have AD and is receiving an additional therapy for AD.

The present invention also provides a kit comprising the compound of the invention, or a pharmaceutically acceptable salt, tautomer, solvate, hydrate, prodrug, derivative, stereoisomer, analog or isotopically labelled derivative thereof, or a composition of the invention, for use in the treatment and/or prevention of protein misfolding disease and/or a neurodegenerative disease as described above. The kit optionally further comprises, in admixture or in separate containers, an additional pharmaceutically active agent(s) as described above. Preferred features of the compound or composition for use as defined herein are also preferred features of the kit of the invention.

### EXAMPLES

### Methods—In Vitro

### Preparation of Aβ Peptides

The recombinant Aβ(M1-42) peptide (MDAEFRHDSGYEVHHQKLVFFAEDVG-SNKGAIIGLMVGGVVIA [SEQ ID NO: 1]), here called Aβ42, was expressed in the E. coli BL21 Gold (DE3) strain (Stratagene, Calif., U.S.A.) and purified as described previously with slight modifications. Briefly, the purification procedure involved sonication of E. coli cells, dissolution of inclusion bodies in 8 M urea, and ion exchange in batch mode on diethylaminoethyl cellulose resin and lyophylization. The lyophilized fractions were further purified using Superdex 75 HR 26/60 column (GE Healthcare, Buckinghamshire, U.K.) and eluates were analyzed using SDS-PAGE for the presence of the desired protein product. The fractions containing the recombinant protein were combined, frozen using liquid nitrogen, and lyophilized again.

### Preparation of Small Molecules

Except for Netoglitazone, which was custom synthesised by GVK BIO, all small molecules were purchased with a purity greater than 99%. Small molecules were first solubilized in 100% DMSO to a concentration of 5 mM, and then diluted in the peptide solution to reach a final DMSO concentration of maximum 1-3%. We verified that the addition of DMSO in the reaction mixture has no effect on Aβ42 aggregation.

### Preparation of Samples for Kinetic Experiments

Solutions of monomeric peptides were prepared by dissolving the lyophilized Aβ42 peptide in 6 M GuHCl. Monomeric forms were purified from potential oligomeric species and salt using a Superdex 75 10/300 GL column (GE Healthcare) at a flowrate of 0.5 mL/min, and were eluted in 20 mM sodium phosphate buffer, pH 8 supplemented with 200 μM EDTA and 0.02% NaN₃. The centre of the peak was collected, and the peptide concentration was determined from the absorbance of the integrated peak area using ε₂₈₀=1490 L mol⁻¹ cm⁻¹. The obtained monomer was diluted with buffer to the desired concentration and supplemented with 20 μM Thioflavin T (ThT) from a 1 mM stock. All samples were prepared in low binding Eppendorf tubes on ice using careful pipetting to avoid introduction of air bubbles. Each sample was then pipetted into multiple wells of a 96-well half-area, low-binding, clear bottom and PEG coating plate (Corning 3881), 80 μL per well. Aβ42 kinetics have been performed in the absence or the presence of Netoglitazone, Mitoglitazone, Rosiglotazone, Rivoglitazone, Pioglitazone, Ciglitazone, Englitazone, Darglitazone, Troglitazone and Balaglitazone.

For the seeded experiments, preformed fibrils were prepared just prior to the experiment. Kinetic experiments were set up as described above for 5 μM Aβ42 samples in 20 mM sodium phosphate buffer, pH 8 with 200 W EDTA, 0.02% NaN₃ and 20 μM ThT. The ThT fluorescence was monitored for 3 hours to verify the formation of fibrils. Samples were then collected from the wells into low-binding tubes. Under the considered conditions (i.e. 5 μM Aβ42), the monomer concentration is negligible at equilibrium. The final concentration of fibrils, in monomer equivalents, was considered equal to the initial concentration of monomer. Fibrils were then added to freshly prepared monomer in order to reach either 2% or 50% final concentration of seeds in the absence or the presence of Netoglitazone.

For the experiments of Aβ42 aggregation kinetics in human CSF, monomeric solutions of 3 μM Aβ42 were prepared similar to above with the only exception that the buffer was 20 mM Hepes, pH 8 supplemented with 1 mM CaCl₂ at 150 mM NaCl. The obtained monomer was diluted with the buffer in order to reach 66% final concentration of CSF, in which the effect of CSF is close to maximum. Aβ42 aggregation kinetics were performed in the absence and the presence of 1.25 and 5-fold excess of Netoglitazone.

For the experiments monitoring Aβ40 aggregation kinetics, the experiments were performed similarly to those described above for Aβ42 at a concentration of 10 μM of Aβ40 in the absence or presence of 1.25-fold excess of Netoglitazone.

### Kinetic Assays

Assays were initiated by placing the 96-well plate at 37° C. under quiescent conditions in a plate reader (Fluostar Omega, Fluostar Optima or Fluostar Galaxy, BMGLabtech, Offenburg, Germany). The ThT fluorescence was measured through the bottom of the plate with a 440 nm excitation filter and a 480 nm emission filter. The ThT fluorescence was followed for three repeats of each sample.

### Theoretical Analysis

The time evolution of the total fibril mass concentration is described as a function only of the initial conditions and the rate constants of the system by the integrated rate law given by Eq. (54) in Cohen et al., J Chem Phys 135, 065106, 2011.

Interestingly, to capture the complete assembly process for Aβ42 (Cohen et al., Proc Natl Acad Sci USA, 110(24), 9758-63, 2013), only two particular combinations of the rate constants define much of the macroscopic behaviour. These are related to the rate of formation of new aggregates through primary pathways λ={square root over (2k₊kₙm(0)ⁿ^{_{c}})} and through secondary pathways κ=\sqrt{2k₊k₂m(0)ⁿ^{₂}⁺¹}, where the initial concentration of soluble monomers is denoted by m(0), n_{c} and n₂ describe the dependencies of the primary and secondary pathways on the monomer concentration (n_{c}=n₂=2 for Aβ42), and kₙ, k₊ and k₂ are the rate constants of the primary nucleation, elongation and secondary nucleation, respectively (Cohen et al., Proc Natl Acad Sci USA, 110(24), 9758-63, 2013). For Aβ42, under the conditions considered here (i.e. micromolar concentrations of Aβ42), the rate of depolymerisation is significantly less than the rate of fibril elongation throughout the reaction time course (i.e. until the monomeric peptide is almost entirely depleted) and hence this process can be neglected in the kinetic analysis.

Inhibitors can interfere with the aggregation process by inhibiting one or more of the individual microscopic steps. We can identify the microscopic events that are inhibited by the chemical compounds by fitting the integrated rate law (Eq. (54) in Cohen et al., J Chem Phys 135, 065106) to the macroscopic aggregation profiles and comparing the fitted set of microscopic rate constants (k₊k₂ and k₊kₙ in the absence of pre-formed seeds; k₊ and k₂ in the presence of pre-formed seeds where primary nucleation is bypassed) required to describe the time evolution of the fibril formation in the absence and presence of Netoglitazone. The analysis is analogous to that carried out in Habchi et al., Proc Natl Acad Sci USA; 114(2):E200-E208, 2017 to study the effects of other small molecules on Aβ42 aggregation.

Using the rate constants (kₙ, k₂ or k₊) in the presence of the molecules, we can also estimate the reactive flux towards oligomers (r(t)) as:

r(t)=kₙm(t)ⁿ^{_{c}}+k₂m(t)ⁿ^{₂}M(t) (Eq. 1)

The time at which the generation of oligomers reaches a peak, as well as the total number of oligomers generated over time (time integral of r(t)) can subsequently be predicted.

### Dot Blot Assay

Blotting was performed using Aβ42 fibril-specific antibody (OC, Millipore). During the time course of the aggregation of a 2 μM Aβ42 in the absence and in the presence of 5-fold excess of Netoglitazone, 4 μL Aβ42 aliquots were removed from the mixture at different time points for blotting with OC. Aβ42 aliquots were spotted onto a nitrocellulose membrane (0.2 μm, Whatman) and then the membranes were dried and then blocked with Blocking One (Nacalai tesque) before immuno-detection. OC was used according to the manufacturer's instructions. Alexa Fluor® 488-conjugated secondary antibodies (Life technologies) were subsequently added and fluorescence detection was performed using Typhoon Trio Imager (GE Healthcare).

### ELISA-Based Binding of Oligomer-Specific Antibodies

20 μl Aliquots were taken at the t₅₀ (i.e. half-time) from aggregation reactions of 5 μM Aβ42 in the absence and in the presence of 5-fold excess of Netoglitazone. Samples were then immobilised on a 96-well Maxisorp ELISA plate (Nunc, Roskilde, Denmark) with no shaking for 1 h at room temperature. The plate was then washed three times with 20 mM Tris pH 7.4, 100 mM NaCl and incubated in 20 mM Tris pH 7.4, 100 mM NaCl, 5% BSA under constant shaking overnight at 4° C. The day after the plate was washed six times with 20 mM Tris pH 7.4, 100 mM NaCl and then incubated with 30 μl solutions of 5 μM oligomer-specific antibody under constant shaking either for 1 hour or overnight at room temperature. At the end of this incubation, the plate was washed six times with 20 mM Tris pH 7.4, 100 mM NaCl and incubated with 30 μl solutions of Rabbit polyclonal to 6× His Tag® HRP conjugated (Abcam, Cambridge, UK) at a dilution of 1:4000 in 20 mM Tris pH 7.4, 100 mM NaCl, 5% BSA under shaking for 1 hour at room temperature. The plate was washed 3 times with 20 mM Tris pH 7.4, 100 mM NaCl, then twice with 20 mM Tris pH 7.4, 100 mM NaCl, 0.02% Tween-20 and again three times with 20 mM Tris pH 7.4, 100 mM NaCl. Finally, the amount of bound oligomer-specific antibody was quantified by using 1-Step™ Ultra TMB-ELISA Substrate Solution (ThermoFisher Scientific, Waltham, Mass., United States), according to manufacturer instructions, and measuring the absorbance at 450 nm by means of a CLARIOstar plate reader (BMG Labtech, Aylesbury, UK).

### Ca²⁺ Influx Assay

Single vesicles tethered to PLL-PEG coated borosilicate glass coverslides (VWR International, 22×22 mm, product number 63 1-0122) were placed on an oil immersion objective mounted on an inverted Olympus IX-71 microscope. Each coverslide was affixed at Frame-Seal incubation chambers and was incubated with 50 μL of HEPES buffer of pH 6.5. Just before the imaging, the HEPES buffer was replaced with 50 μL Ca²⁺ containing buffer solution L-15. 16 (4×4) images of the coverslide were recorded under three different conditions (background, in the presence of Aβ42 and after addition of ionomycin (Cambridge Bioscience Ltd, Cambridge, UK), respectively). The distance between each field of view was set to 100 μm, and was automated (bean-shell script, Micromanager) to avoid any user bias. After each measurement the script allowed the stage (Prior H117, Rockland, Mass., USA) to move the field of view back to the start position such that identical fields of view could be acquired for the three different conditions. Images of the background were acquired in the presence of L15 buffer. For each field of view 50 images were taken with an exposure time of 50 ms. Thereafter, 50 μL of the aggregation reaction, diluted to a concentration of twice the targeted value, was added and incubated for 10 min. Next, 10 μL of a solution containing 1 mg/mL of ionomycin (Cambridge Bioscience Ltd, Cambridge, UK) was added and incubated for 5 min and subsequently images of Ca²⁺ saturated single vesicles in the same fields of view were acquired. The recorded images were analysed using ImageJ to determine the fluorescence intensity of each spot under the three different conditions in the presence of an aggregation mixture incubated with and without Netoglitazone.

### Methods—In Vivo (C. Elegans)

### Media Preparation

Standard conditions were used for the propagation of C. elegans (S. Brenner, The genetics of Caenorhabditis elegans. Genetics. 77, 71-94 (1974)). Briefly, the animals were synchronized by hypochlorite bleaching, hatched overnight in M9 buffer (3 g/l KH₂PO₄, 6 g/l Na₂HPO₄, 5 g/l NaCl, 1 μM MgSO₄), and subsequently cultured at 20° C. on nematode growth medium (NGM) (CaCl₂) 1 mM, MgSO₄ 1 mM, cholesterol 5 μg/mL, PBS Buffer (250 μM KH₂PO₄, 67.5 μM KCl, 3.425 mM of NaCl, pH 6), Agar 17 g/L, NaCl 3 g/1, casein 7.5 g/l) plates seeded with the E. coli strain OP50. Saturated cultures of OP50 were grown by inoculating 50 ml of LB medium (tryptone 10 g/l, NaCl 10 g/1, yeast extract 5 g/l) with OP50 and incubating the culture for 16 h at 37° C. NGM plates were seeded with bacteria by adding 350 μl of saturated OP50 to each plate and leaving the plates at 20° C. for 2-3 days. On day 3 after synchronisation, the animals were placed on NGM plates containing 5-fluoro-2′deoxy-uridine (FUDR) (75 μM, unless stated otherwise) to inhibit the growth of offspring.

### Strains

The following strains were used:

GMC101 dvIs100 [unc-54p::A-beta-1-42::unc-54 3′UTR+mtl-2p::GFP]. mtl-2p::GFP produces constitutive expression of GFP in intestinal cells. unc-54p::A-beta-1-42 expresses full-length human Aβ42 peptide in body wall muscle cells that aggregates in vivo. Shifting L4 or young adult animals from 20° to 24° C. causes paralysis (G. McColl et al., Utility of an improved model of amyloid-beta (Aβ₁₋₄₂) toxicity in Caenorhabditis elegans for drug screening for Alzheimer's disease. Mol Neurodegener. 7, 57 (2012));

NL5901 (pk1s2386 [α-synuclein::YFP unc-119(+)]) (PD worms), in which α-synuclein fused to YFP relocates to inclusions, which are visible as early as day 2 after hatching and increase in number and size during the aging of the animals, up to late adulthood (day 17) (T. J. Van Ham et al., C. elegans model identifies genetic modifiers of α-synuclein inclusion formation during aging. PLoS Genetics. 4 (2008));

CL2331; dvIs37 [myo-3p::GFP::A-Beta (3-42)+rol-6(su1006)] (Aβ₃₋₄₂::GFP_{Muscular} worms). Maintain at 16C. Roller. Diffuse and aggregated GFP expression in body wall muscle. Low brood size. Sicker at higher temperatures. (C. D. Link et al., The β amyloid peptide can act as a modular aggregation domain. Neurobiol. Dis. 32, 420-425 (2008)); and

CL2355 [pCL45 (snb-1::Abeta 1-42::3' UTR(long)+mtl-2::GFP] (Aβ_{1-42Neur} worms). Maintain at 16C. Pan-neuronal expression of human Abeta peptide. Constitutive intestinal expression of GFP from marker transgene. Strain shows deficits in chemotaxis, associative learning, and thrashing in liquid. Strain also has incomplete sterility due to germline proliferation defects and embryonic lethality (Y. Wu et al., Amyloid-beta-induced pathological behaviors are suppressed by Ginkgo biloba extract EGb 761 and ginkgolides in transgenic Caenorhabditis elegans. J. Neurosci. 26, 13102-13113 (2006)).

N2 C. elegans var. Bristol used as controls (also labelled “healthy”). Generation time is about 3 days. Brood size is about 350, wild type phenotype, sub-cultured in 1973 (S. Brenner, The genetics of Caenorhabditis elegans. Genetics. 77, 71-94 (1974)).

### Drug Administration

Drugs were administered as previously shown (M. Perni et al., Massively parallel C. elegans tracking provides multi-dimensional fingerprints for phenotypic discovery. J. Neurosci. Methods. 306, 57-67 (2018); J. Habchi et al., An anticancer drug suppresses the primary nucleation reaction that initiates the production of the toxic Aβ42 aggregates linked with Alzheimers disease. Science Advances. 2, e1501244-e1501244 (2016); J. Habchi et al., Systematic development of small molecules to inhibit specific microscopic steps of Aβ42 aggregation in Alzheimer's disease. Proc Natl Acad Sci USA. 114, E200-E208 (2017); M. Perni et al., Multistep Inhibition of α-Synuclein Aggregation and Toxicity in Vitro and in Vivo by Trodusquemine. ACS Chem Biol, 17; 13(8):2308-2319 (2018)).

Briefly, Netoglitazone stocks (5 mM in 100% DMSO) were used at an appropriate concentration to seed 9-cm NGM plates. Plates were then placed in a laminar flow hood at room temperature (22° C.) for up to 4 hours to dry. C. elegans cultures were then transferred onto media seeded with compound as L4 stage or Day 3 for late treatments and incubated at 24° for the whole experiment. Experiments were carried out at different Netoglitazone concentrations ranging from 0.05 to 500 μM in 1% DMSO. As controls, plates seeded only with 1% DMSO were used.

### Automated Motility Assay

All C. elegans populations were cultured at 20° C. and developmentally synchronized from a 4 h egg-lay. At 64-72 h post-egg-lay (time zero), individuals were transferred to FUDR plates, and body movements were assessed over the times indicated. At different ages, the animals were washed off the plates with M9 buffer and spread over an OP-50 unseeded 9 cm plate, after which their movements were recorded at 20 fps using a recently developed microscopic procedure (M. Perni et al., Massively parallel C. elegans tracking provides multi-dimensional fingerprints for phenotypic discovery. J. Neurosci. Methods. 306, 57-67 (2018)) for 1 min. Up to 600 animals were counted in each experiment in duplicate unless stated otherwise. One experiment that is representative of the three or more measured in each series of experiments is shown, and videos were analysed using a custom-made tracking code (M. Perni et al., Massively parallel C. elegans tracking provides multi-dimensional fingerprints for phenotypic discovery. J. Neurosci. Methods. 306, 57-67 (2018)).

Staining and Microscopy in Living C. elegans

Plaques staining was carried out as previously described (J. Habchi et al. (2016); M. Perni et al., A natural product inhibits the initiation of α-synuclein aggregation & suppresses its toxicity. Proc. Natl. Acad. Sci. U.S.A. 114, E1009-E1017 (2017)). Briefly, live transgenic animals were incubated with NIAD-4 over a range of concentrations and times, with 1 μM NIAD-4 (0.1% DMSO in M9 buffer) for 4 hours at room temperature. After staining, animals were allowed to recover on NGM plates for about 24 hours to allow destaining via normal metabolism. Stained animals were mounted on 2% agarose pads containing 40 mM NaN₃ as anaesthetic on glass microscope slides for imaging. Images were captured with a Zeiss Axio Observer D1 fluorescence microscope (Carl Zeiss Microscopy GmbH) with a 20× objective and a 49004 ET-CY3/TRITC filter (Chroma Technology Corp). Fluorescence intensity was calculated using ImageJ software (National Institutes of Health) and then normalized as the corrected total cell fluorescence. Only the head region was considered because of the high background signal in the guts. All experiments were carried out in triplicate, and the data from one representative experiment are shown. Statistical significance was determined using t tests.

### Chemotaxis Assay

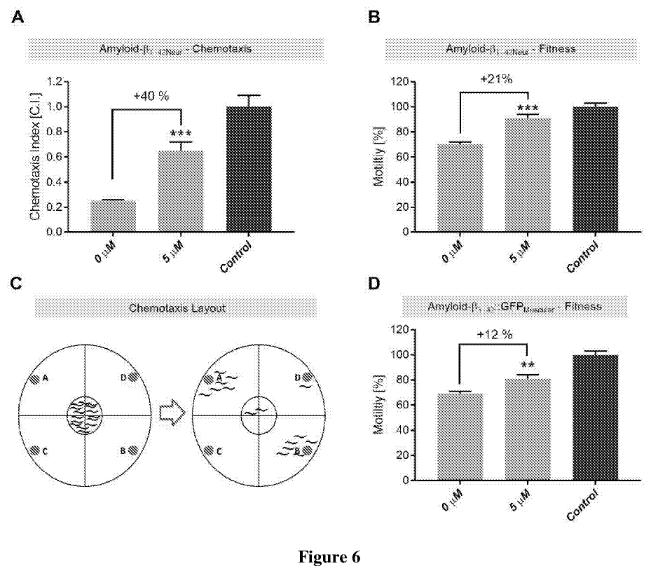

Chemotaxis measurements were carried out as previously described (O. Margie, C. Palmer, I. Chin-Sang, C. elegans Chemotaxis Assay. J Vis Exp, e50069 (2013)) and as illustrated in FIG. 6C. Briefly, adult synchronized transgenic C. elegans CL2355 worms and wild-type healthy worms were incubated with or without 5 μM Netoglitazone for 5 days 24° C. At day 6 of adulthood the worms were then collected, washed with M9 buffer three times, and assayed in 9 cm screening plates (1.9% agar, 1 mM CaCl₂), 1 mM MgSO₄, and 25 mM phosphate buffer, pH 6.0) seeded with 50 μl of a 10× culture of Op50 Bacteria or sterile water, as attractant or test conditions, respectively and in combination with 1 μl of 1M Levamisole. Ca. 200 worms were placed in the central quadrant of the plate and incubated at 24° C. for 8 h, after which the chemotaxis index (CI) was scored. The CI was defined as follows (O. Margie et al (2013)):

(number of worms at the attractant locations−number of worms at the control locations)/total number of worms on the plate

Worms that were remaining in the central quadrant were excluded.

### ROS Production and Measurement

ROS-Glo™ H₂O₂ cell kit assay was used (Promega, Fitchburg, Wis., USA) and adapted for C. elegans studies. The ROS-Glo™ H₂O₂ Assay is a bioluminescent assay that measures the level of H₂O₂, a reactive oxygen species (ROS), directly in cell culture or tissue or in defined enzyme reactions. A derivatized luciferin substrate is incubated with sample and reacts directly with H₂O₂ to generate a luciferin precursor. Worms treated with 5 μM Netoglitazone in 1% DMSO or 1% DMSO only were washed using M9 buffer out the NGM plates. The buffer was then changed 3 times to remove the excess bacteria. Worm pellets were then divided in three wells and 80 μl of worm pellet (around 200 worms/well) was incubated for 6 h at RT with 20 μl of a ROS Substrate Solution (Promega, Fitchburg, Wis., USA); mild shaking at 300 rpm was used to avoid worm sedimentation; afterwards, worms were incubated for ca. 20 min with 100 μl of the detection solution; luminescence was then measured with a Clariostar (BMG Labtech, Aylesbury, UK).

### Methods—In Vivo Efficacy (Mouse)

### APPPS1 Mice

APPPS1 transgenic mice were used in the study, which co-express the Swedish mutation K670M/N671L and PS1 mutation L166P under the control of the neuron-specific Thy-1 promoter on a C57BL/6 genetic background. APPPS1 mice were habituated ahead of the study to voluntarily drink a condensed milk formulation from a pipette. The condensed milk used in the study is commercially available (Migros) and contains milk, sugar, stabilizer E339. Body weight was measured ahead of commencing the study to calculate the dose of Netoglitazone for each mouse and to calculate the total blood volume. Mice aged 60 days old were then dosed once daily with a pipette for 90 days. The pipette contained 40-80 μL consisting of either condensed milk (2 ml/kg/day) only (placebo cohort) or condensed milk (2 ml/kg/day) and Netoglitazone (75 mg/kg/day). Blood was collected via retro-orbital sampling to monitor the concentration of Netoglitazone after 7 days and 28 days. Visual monitoring of the mice and measurement of body weight were conducted daily and every other week respectively. At the end of the experiment (i.e. after 90 days of once daily treatment such that mice were 150 days old), mice were euthanized and their brains were analyzed as outlined below.

### Perfusions of APPPS1 Mice

The A4B4P4 hydrogel formulation (Chung, K. et al. 2013) used consisted of an aqueous solution of 4% Acrylamide (wt/vol), 0.05% Bis-Acrylamide (wt/vol), 4% Paraformaldehyde (wt/vol), and 0.25% VA-044 initiator (wt/vol) in PBS. Animals were deeply anesthetized and perfused transcardially with phosphate buffered saline solution (PBS, ThermoFisher, pH 7.4), followed by perfusion of an equal volume of cold A4B4P4. As a result, tissue is sufficiently crosslinked to maintain structural stability. To embed tissues into hydrogel, tissues were polymerized following the nitrogen-flush or vacuum chamber degassing protocol previously described (Chung, K. et al. 2013). All samples were polymerized by incubation in a 37° C. shaking incubator for 2.5 hours, followed by removal of excess hydrogel. Solid gels can be peeled from the sample under a fume hood and disposed as solid waste. By perfusing mice in vivo with hydrogel solutions, all nucleic acids and proteins are fixed in place. Samples were then placed in clearing solution of 8% sodium dodecyl sulfate (SDS) in Sodium Borate buffer (200 mM, pH 8.5) at 37° C. and actively cleared with the method described below.

### Clearing

A tissue clearing method was used whereby tissue blocks or whole organs are rendered transparent and are hence amenable to whole-brain imaging. An electrophoretic field of 130 mA, 60V and 15W was applied at 37° C. for several hours to improve clearing of the lipids until optical transparency was achieved. Before further processing, clearing solution were rinsed from the sample with 2-3 washes of PBST (0.1% TritonX-100 (wt/vol) in PBS) over 1 day.

### Histochemistry of Whole Brains

Cleared whole brains were washed three times in PBS for 1 h and once overnight. Brains were then incubated for 1 h in 1×TTB (1M Tris, 1M Tricine, pH 8.5). Brains were stained with luminescent conjugated polythiophenes (LCPs) to detect Aβ deposits for 2 h at room temperature. LCPs were diluted 1:100 in 1% Low Melting Point Agarose (LMA). The LMA was applied on the brain until it reached the solidified phase. An electric field of 20V, 20 mA was applied to move the LCPs through the tissue from the negative to the positive pole. Samples were washed several times in PBS at room temperature following the staining.

### Imaging of Samples

To prepare samples for imaging, samples were incubated in a refractive index matching solution (Histodenz) for 2 days. Samples were mounted in UQ-753 40×40 cuvettes (Portman Instruments). Samples were imaged using a custom-made meso-SPIM microscope, using a 2× objective, at 3 μm z-step resolution. Entire samples were obtained by stitching 32 z-stacks tile images with TeraStitcher software and viewed in ImageJ and Imaris 8 (Bitplane).

### Mouse Brain Data Analysis

The image files were analysed using custom-coded software. Each file corresponded to a brain hemisphere and consisted of multiple 2D slices (approximately 2500 slices per hemisphere), with a slice being a focal plane. The files were read one plane at a time with each plane analysed separately. The detection of the plaques was computed using the Laplacian of Gaussians method: a convolution between the Laplacian operator applied to a discretized Gaussian kernel and the image in the original format (16-bit) was performed. Given the overall globular shape of the plaques, the parameter σ of the Gaussian was kept constant in both x and y directions, without loss of generality, in order to enhance the speed of the analysis. The algorithm was free to vary this parameter from a minimum of 1σ to a maximum of 4σ (given the maximum size of the plaques), with 2 steps between the two values. The overlap threshold value of 0.5 was set, in order to merge plaques whose area would show a larger overlap than the threshold, and to avoid overcounting. Upon the identification of the local minima, corresponding to the centres of the plaques, the plaques were then counted. The total area can also be computed by integrating the region contained within the zero-crossing points of the function with the plane. The total number (area) of plaques per hemisphere was the sum of the plaques (areas) detected on each plane.

### Mouse Brain Data Analysis Code Specifics

Language—Python 3.6.8

Libraries—Numpy, Scipy, Opencv, Scikit-image, Matplotlib, Tifffile, Seaborn

Custom modules: my_imaging (included in the package)

### Mouse Brain Data Analysis Code Implementation

import matplotlib.pyplot as plt
import numpy as np
import cv2

import my_imaging
from skimage import morphology as mpy
from skimage import measure, exposure, filters
from skimage.feature import blob_log
from skimage.segmentation import inverse_gaussian_gradient
import time
import datetime
import seaborn as sns
from astropy.stats import median_absolute_deviation as mad
import sys
from tifffile import TiffFile
import argparse
from scipy import ndimage
from skimage import img_as_ubyte as img_as_ub
def bg_threshold(im, multiplier=1.):

bg_intensity = np.bincount(np.ravel(np.array(im, dtype=int)))[:−1].argmax( )
return multiplier*bg_intensity-im.min( )

#input variables − input parser
ap = argparse.ArgumentParser( )
ap.add_argument(“−i”, “--image”, required=True, help=“path to input image”)
args = vars(ap.parse_args( ))
# TIFFFILE COMMANDS TO LOAD
brain = TiffFile(args[“image”])
fname = args[“image”]
h, w = brain.pages[0].shape[0], brain.pages[0].shape[1]
total_pxls = w * h
mult = 2.0 # used to define the ROIs
print_chkp = 500 # when to print checkpoints
start_idx = 0 # this can change with the index of the for loop to restart the analysis at a
different plane
rep_file = open(f“Report_Startplane_{ start_idx}.txt”, mode=‘w’, buffering=1)
start = time.time( )
rep_file.write(f“===== Header =====\n”

f“Analysis started at: {datetime.datetime.now( )}\n”
f“Reading file: {fname}\n”
f“Plane sizes: Height {h} − Width {w}\n”
f“Number of Slices: {len(brain.pages)}\n”
f“Images are low-contrast: {exposure.is_low_contrast(brain.pages[0].asarray( ))}\n”
f“Multiplier background detection: {mult}\n”
f“Outfile: {rep_file.name}\n”
f“==================\n\n”)

rep file.write(“===== Results [Legend] =====\n”

“Plane: index of the img slice\n”
“#Plqs: number of plaques found\n”
“Plqs_cs: Total plaques cross-section\n”
“=============================\n”)

rep_file.write(f“\n#Plane\t#Plqs\tPlqs_cs\n”)
fig = plt.figure(figsize=(20, 10)) # figure to print checkpoints
plq_count = 0 # plaques count
plq_cs = 0 # plaques cross section
for pln in range(len(brain.pages)):

if pln != 0 and not pln % 10:

print(f“Processing Plane {pln} − Time elapsed: {round((time.time( )-start)/60, 2)}

minutes\n”)

img = brain.pages[pln].asarray( ) # tifffile
“““Detect plaques with Laplacian of Gaussian method and count areas”””
blobs_log = blob_log(img, max_sigma=4, min_sigma=1, threshold=0.01. overlap=0.5)
blobs_log[:, 2] = blobs_log[:, 2] * np.sqrt(2)
equiv_area = round(sum(np.pi * blobs_log[:, 2]**2))
if equiv_area:

plq_cs += equiv_area

plq_count += len(blobs_log)
rep_file.write(f“{pln}\t{len(blobs_log)}\t{int(equiv_area)}\n”)
if pln != 0 and not pln % print_chkp:

print(f“Printing checkpoint image − Plane {pln}”)
thr = my_imaging.bg_threshold(img, multiplier=mult)
mask = img > thr
mask = mpy.binary_opening(mask, mpy.square(10))
mask = mpy.binary_closing(mask, mpy.square(5))
img_masked = img * mask
img_masked_bin = img_masked > 0
labels = measure.label(img_masked_bin, background=0)
props = measure.regionprops(labels)
log_img = np.log2(img + np.ones(img.shape)) # for visualisation purpose
“““Plotting”””
ax = plt.subplot(1, 3, 1)
ax.set_title(“Logged Intensities”)
plt.imshow(log_img)
ax = plt.subplot(1, 3, 2)
ax.set_title(f“Laplacian of Gaussian\nPlaques: {len(blobs_log)}”)
plt.imshow(log_img)
for blob in blobs_log:

y, x, r = blob
c = plt.Circle((x, y), r, color=‘red’, linewidth=1, fill=False)
ax.add_patch(c)

ax = plt.subplot(1, 3, 3)
ax.set_title(“ROIs”)
plt.imshow(labels)
plt.savefig(f“{pln}_plane_plaques.pdf”, bbox_inches=“tight”)
plt.cla( )

del img
del blobs_log
if pln != 0 and not pln % print_chkp:

del thr
del mask
del img_masked
del img_masked_bin
del labels
del props
del log_img

rep_file.write(f“===== Summary =====:\n”

f“Analysis finished at: {time.time( )}\n”
f“Plaques_number: {plq_count}\n”
f“Plaques_area: {plq_cs}\n”
f“===================”)

print(f“===== Summary =====:\n”

```
  f“Plaques_number: {plq_count}\n”
  f“Plaques_area: {plq_cs}\n”
  f“===================”)
```

rep_file.close( )

### Experimental Examples

The experimental anti-diabetic drug Netoglitazone is a peroxisome proliferator-activated receptor (PPAR) agonist belonging to the thiazolidinedione group. The present inventors have confirmed the effect of Netoglitazone and other glitazones using a range of biochemical, biophysical tools, including measurements in human Cerebrospinal fluid (CSF) and using an in vivo model of AD based on an Aβ-mediated toxicity mechanism, Caenorhabditis elegans (C. elegans). Characterized by its simple anatomy, short lifespan, and well-established genetics, the nematode worm Caenorhabditis elegans has become a powerful model organism in biomedical research, in particular for genetic studies and drug screening. These worms are small (ca. 1 mm in length), transparent, easy to manipulate, with a short maturation period of 3 days from egg to adult at 25° C., and a life-span between 2 and 3 weeks, characteristics which facilitate the rapid study of multiple aspects of their biology. Nevertheless, they have a cellular complexity and tissue-specific protein expression profile comparable to that of higher organisms. As a result, C. elegans is commonly employed as a model organism for the characterization of the molecular mechanisms underlying neurodegeneration, in particular protein aggregation.

The health and fitness of C. elegans has conventionally been quantified in liquid media by counting the number of body bends per minute (BPM), or by measuring the speed of movement of the worms. Other key readouts in such studies are lifespan and paralysis which have, for example, recently led to major discoveries in the field of ageing, including the identification of specific genes and compounds modulating longevity, the link between oxidative stress and mitochondrial function, and the triggers for neurodegenerative diseases.

In order to screen for the effect of therapeutics in the most robust way, a wide field-of-view nematode-tracking platform (WF-NTP) was used, which enables the simultaneous investigation of multiple phenotypic readouts on large worm populations. The WF-NTP monitors up 5000 animals in parallel, and the phenotypical readout includes multiple parallel parameters.

It is shown that certain glitazones, including in particular Netoglitazone, are able restore the phenotype of healthy control worms in terms of their fitness and ROS production but not the cognate α-synuclein-mediated toxicity PD model, thus suggesting their specificity towards the aggregation of the Aβ peptide. Finally, it is shown that the improvement that was observed in the fitness of the AD worms correlates extremely well with the decrease in the amount of aggregates that are formed in the worms during their life cycle.

The following non-limiting Examples illustrate the invention.

### Example 1—Netoglitazone Inhibits Aβ Aggregation in a Concentration-Dependent Manner

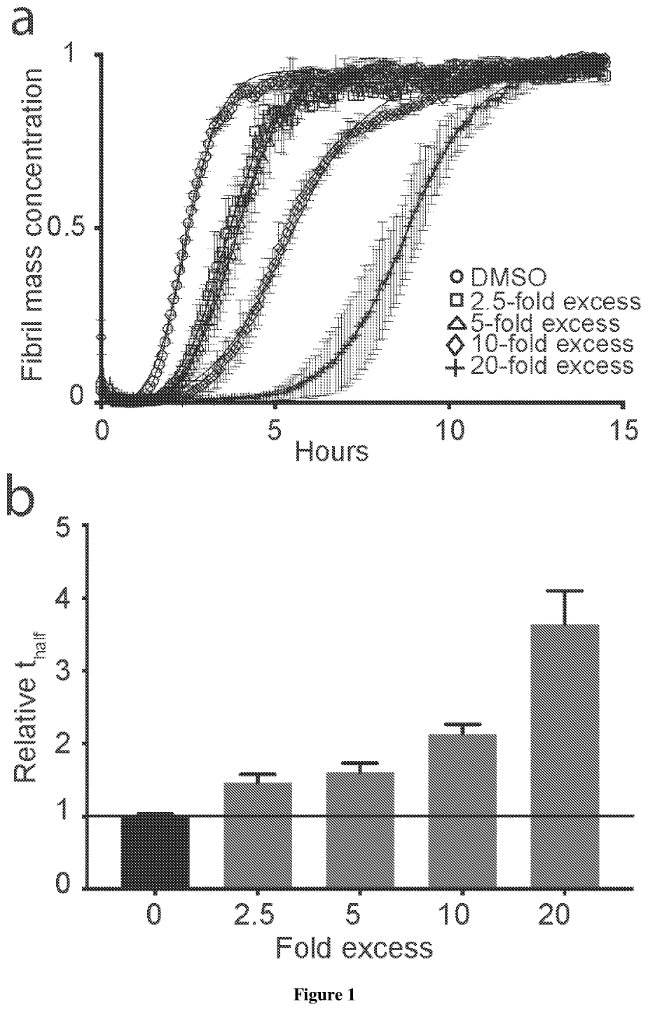

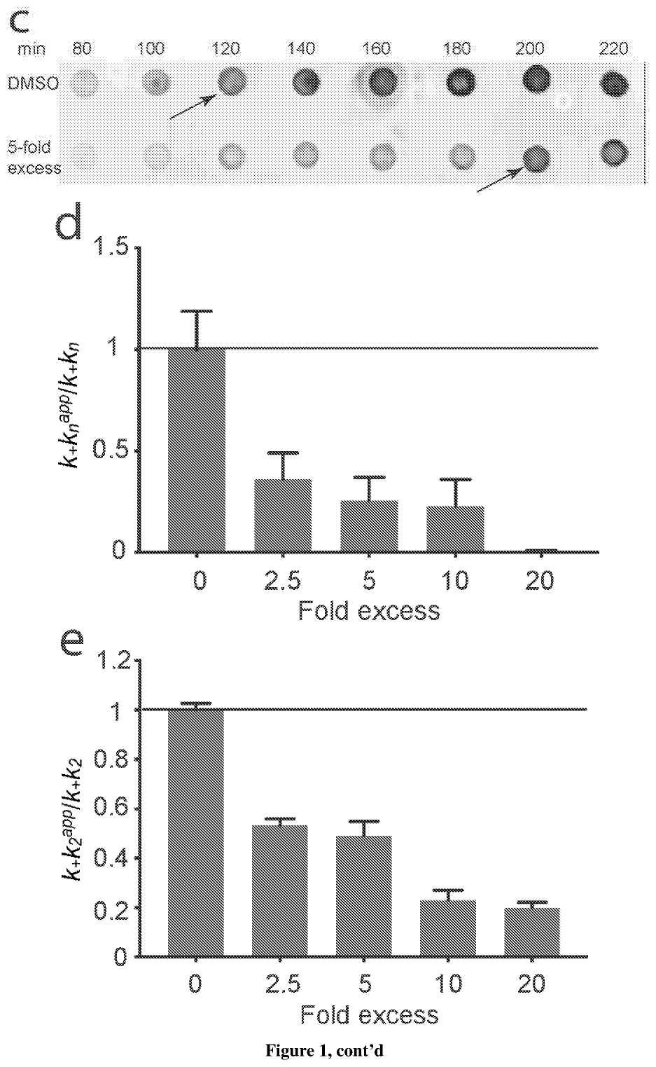

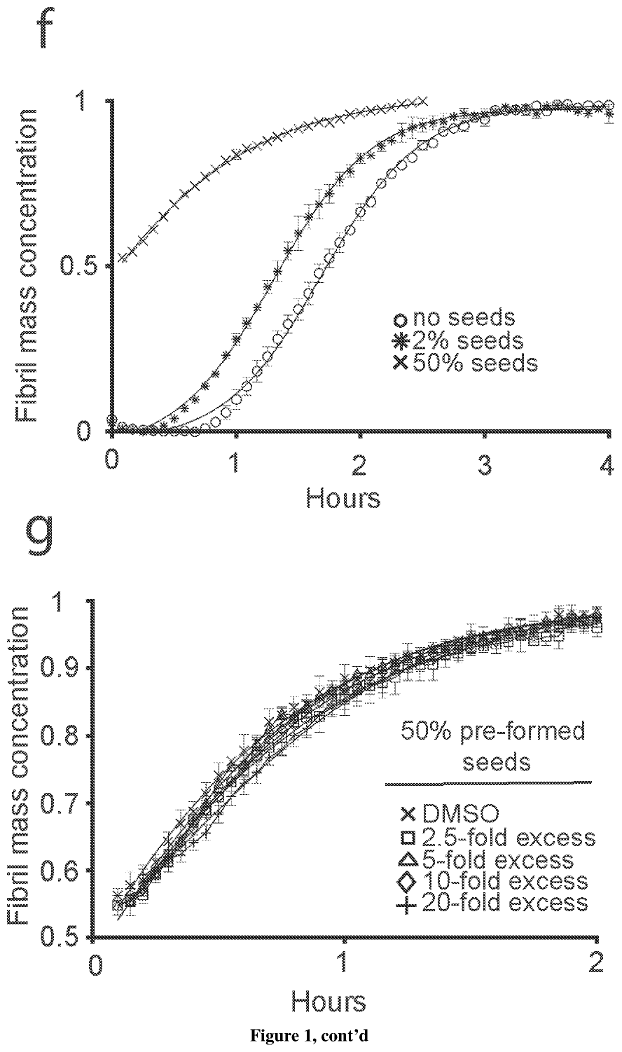

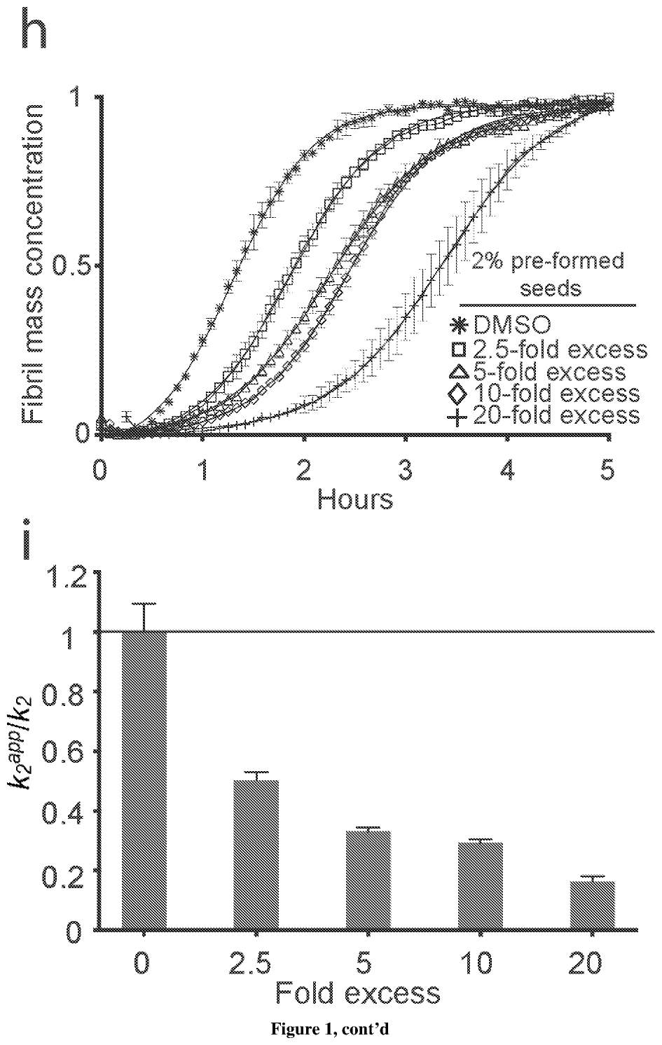

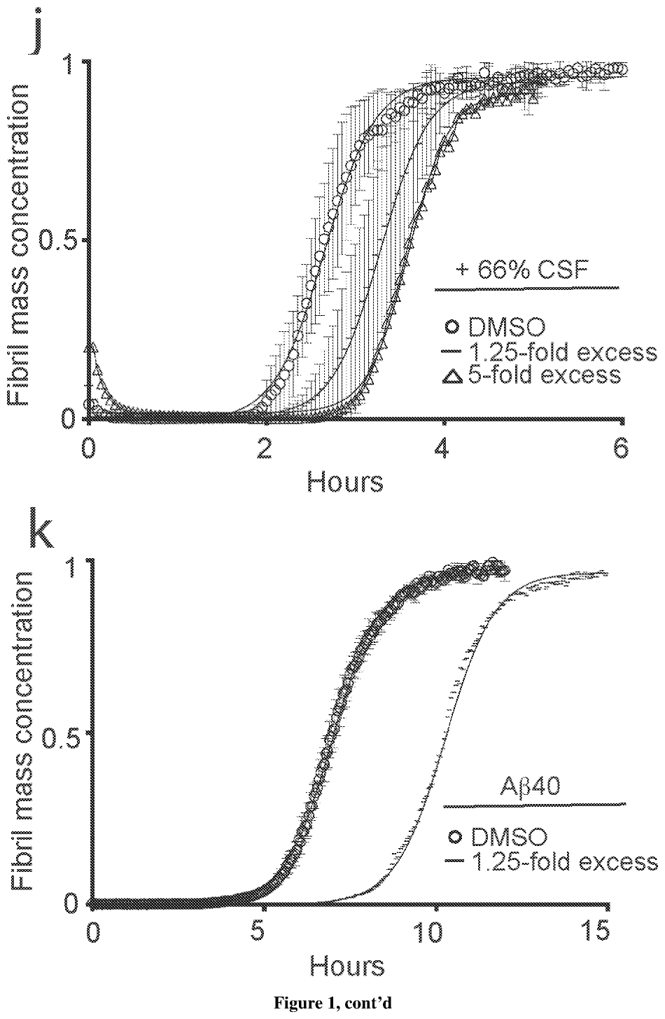

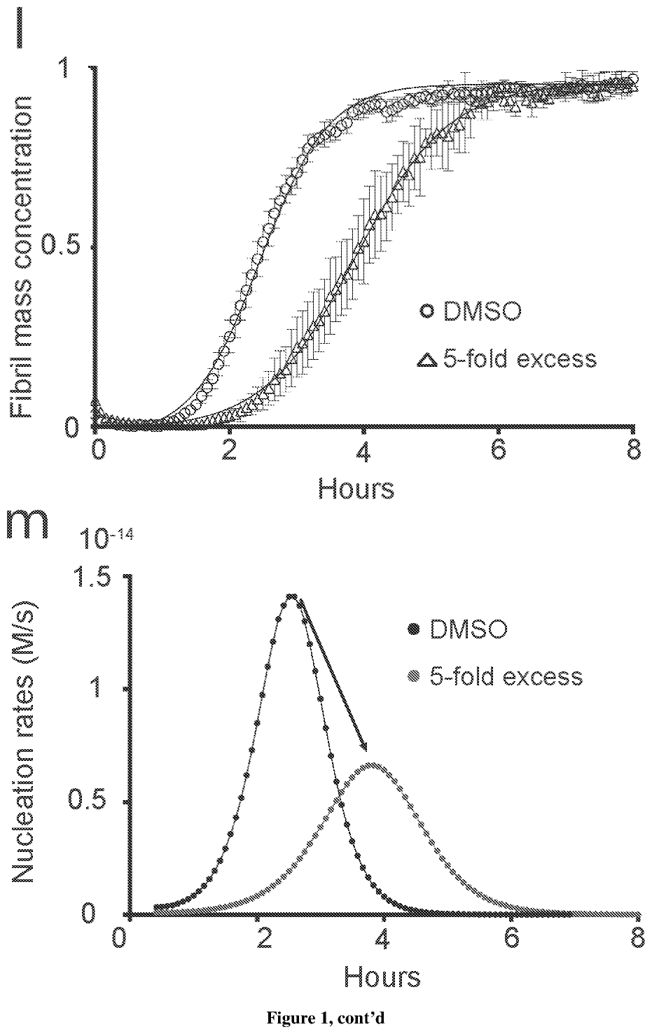

Aβ42 fibril formation was monitored in vitro using a 2 μM Aβ42 sample in the absence and the presence of Netoglitazone. For Aβ42 alone the half-time of aggregation was roughly 2 h under the buffer conditions used. A substantial delay in Aβ42 aggregation was observed in a concentration-dependent manner. This can be seen in FIGS. 1a and 1b.

To investigate these effects further and to exclude possible interferences of the compounds with ThT binding to Aβ42 fibrils and the fluorescence measurements, the quantities of Aβ42 fibrils were probed at eight time points during the aggregation reaction in the absence and presence of 5-fold excess of Netoglitazone using a dot-blot assay with fibril-sensitive OC primary antibodies. These results can be seen in FIG. 1c. The delay induced by Netoglitazone in the dot blot assay was found to be identical within experimental error to that observed in the ThT-based assay.

### Example 2—Netoglitazone Inhibits Primary and Secondary Pathways

A quantitative analysis was carried out on the effects of the molecules by matching the experimental aggregation profiles to kinetic curves calculated using the rate laws derived from a master equation that relates the time evolution of fibril formation to the rate constants of the different microscopic events. In this approach, the aggregation profiles in the presence of an inhibitor are described by introducing into the rate laws suitable perturbations to each of the microscopic rate constants evaluated in the absence of the inhibitor. The modifications of the rate constants required to describe the aggregation profiles in the presence of different concentrations of inhibitor are then indicative of the specific process affected by the presence of Netoglitazone.

In the presence of small molecules, the data are extremely well described when the rate constants of both primary (kₙk₊) and secondary (k₂k₊) pathways are reduced, where kₙ is the rate constant of primary nucleation, k₂ is the rate constant of surface-catalyzed secondary nucleation and k₊ is the rate constant of elongation. All kinetic curves were compared to simulations where both primary and secondary pathways were decreased concomitantly and the rate constants of both pathways were plotted against the concentration of small molecules. These results can be seen in FIGS. 1d and 1e. This analysis reveals that Netoglitazone can affect both nucleation pathways in Aβ42 aggregation to different extents. The increase in the ThT fluorescence at the end of the reaction was examined and similar values were found in all cases. These results suggest that a similar fibril mass concentration is formed irrespective of whether the small molecules are present or not, in agreement with the dot-blot assay. Given that the concentration of the peptide is much lower in vivo, one would expect that a much lower concentration of the drug is required to affect the rate constants of Aβ42 aggregation to the same extent.

### Example 3—Netoglitazone Blocks the Catalytic Cycle of Aβ Aggregation

To further explore the effects of Netoglitazone on distinct steps of the aggregation reaction, specifically the surface-catalyzed secondary nucleation and elongation steps, an additional series of kinetic measurements were carried out in the presence of Netoglitazone and either 2% or 50% of pre-formed fibril seeds. Normalised kinetics profiles in the absence of Netoglitazone under these conditions can be seen in FIG. 1f. For 50% preformed fibrils, the primary and secondary nucleation steps are bypassed and the formation of mature fibrils is greatly accelerated by elongation reactions promoted by the fibril seeds. Under these conditions, Netoglitazone did not affect the aggregation kinetics of 2 μM Aβ42 even at a concentration of 20-fold excess relative to the peptide. This can be seen in FIG. 1g and strongly indicates that Netoglitazone has no effect on elongation.

To obtain a more complete assessment on the effect of Netoglitazone on the secondary pathways of Aβ42 aggregation, the aggregation kinetics of a 2 μM Aβ42 sample in the presence of 2% fibril seeds was measured. These results can be seen in FIG. 1h. Simulations based on the experimental kinetic curves show that primary nucleation is completely bypassed when even the smallest ratios (1%) of pre-formed seeds are introduced in the solution. By contrast, surface-catalyzed secondary nucleation and elongation contribute in different ways to the overall kinetics, with the contribution of elongation becoming more significant with increasing seed concentrations. Hence, following the aggregation kinetics of Aβ42 using different seed concentrations allows the decoupling of the reaction pathway into the surface-catalyzed secondary nucleation and elongation steps. This is crucial in order to characterize the effect of the small molecules at a single microscopic step level that might not otherwise be detected directly from the aggregation kinetics in the absence of preformed seeds. Data at 2% seeds showed a concentration-dependent inhibition of secondary pathways (i.e. reduction of k₂k₊) of Aβ42 aggregation in the presence of Netoglitazone. This can be seen in FIG. 1h. In this case, the decrease could be attributed solely to a decrease in the rate constant of the surface-catalyzed secondary nucleation, i.e. k₂, since no effect could be observed on the elongation of the fibrils, i.e. k₊, at fold excess as high as 20. This can be seen in FIG. 1g. The rate constants could be derived quantitatively from the kinetic curves and were found to be decreased by about 80% in the presence 20-fold excess of Netoglitazone, as shown in FIG. 1i.

### Example 4—Netoglitazone Delays Aβ42 Aggregation Ex Vivo and Inhibits the Aggregation of the 40-Residue Isoform of Aβ, Aβ40

Whether Netoglitazone retards Aβ42 aggregation under more physiologically relevant conditions was explored. Thus, the effect of Netoglitazone on the aggregation kinetics of Aβ42 in human cerebrospinal fluid (CSF) was monitored. CSF caused a concentration-dependent retardation of Aβ42 aggregation, suggesting that Aβ42 aggregation is slower in this fluid in line with previous results. We then investigated the effect of Netoglitazone under conditions where the effect of CSF is close to maximal, i.e. 66%. As can be seen in FIG. 1j, under these conditions Netoglitazone significantly delayed the aggregation kinetics in a concentration-dependent manner similar to what has been observed in buffer. To further investigate the effect of Netoglitazone on the aggregation of the Aβ peptide, similar kinetics experiments were performed on the 40-residue isoform, Aβ40. Interestingly, it was found that Netoglitazone is able to inhibit Aβ40 aggregation similarly to the 42-residue isoform, as shown in FIG. 1k.

Example 5—Netoglitazone inhibits the formation of neurotoxic oligomers and protects against their effect in disrupting lipid membranes. To translate these findings into the possible effects on the generation of toxic forms of Aβ42 oligomers, a combination of simulation and experimental tools were used to assess the effect of Netoglitazone on the formation of Aβ42 oligomers. Indeed, from the aggregation kinetics curves of a 2 μM sample of Aβ42 in the absence or presence of 5-fold excess of Netoglitazone, shown in FIG. 1l, the total rate of formation of oligomers from both primary and secondary processes were simulated. Decreasing the rate of both primary and secondary nucleation is predicted to decrease significantly the total load of toxic oligomers generated during the aggregation reaction. In agreement with this prediction, the simulations show that inhibiting the primary and secondary nucleation steps in Aβ42 aggregation by Netoglitazone is accompanied by a significant delay in the formation of oligomers and a decrease in their number. These results can be seen in FIGS. 1m and 1n. This is expected to lead to a decreased toxicity of the Aβ species that are formed during the aggregation reaction in the presence of Netoglitazone. However, the characterization and quantification of the toxic intermediate species formed during the aggregation process of Aβ is very challenging because of the transient nature of these species. In order to address this problem experimentally, a recently developed ultrasensitive assay (Flagmeier, P., De, S., Wirthensohn, D., Lee, S. F., Vincke, C., Muyldermans, S., Knowles, T. P., et al. (2017). Ultrasensitive Measurement of Ca(2+) Influx into Lipid Vesicles Induced by Protein Aggregates.. Angewandte Chemie—International Edition, 56 (27), 7750-7754. https://doi.org/10.1002/anie.201700966) that allows the measurement of Ca²⁺ influx into lipid vesicles that are disrupted by protein aggregates was used. Indeed, a wide range of experimental evidence suggest that a key mechanism of aggregate-induced cellular damage is the non-specific cell membrane disruption, a process observed in neuronal cells. Interestingly, based on these experiments, the simulations from the kinetics curves were found to be consistent with the measurements using the lipid disruption assay. Indeed, the simulations shown in FIGS. 1m and 1n, which were derived from the kinetics in FIG. 1l, suggest that the delay in the aggregation of Aβ42 that is induced by the presence of Netoglitazone at 5-molar equivalents is expected to decrease the number of oligomers. Interestingly, experimental data obtained from the lipid disruption assay when 5-fold excess of Netoglitazone was added to a 2 μM Aβ42 solution at time 0 h showed, consistent with simulations, that Netoglitazone protected against the neurotoxic species-induced vesicle disruption. Indeed, samples removed from the aggregation reaction of Aβ42 at 0 h and 2 h (the half-time to aggregation completion in the absence of Netoglitazone) showed a significant difference in the effect of the formed species on disrupting lipid membranes, as shown in FIG. 1o. This is in agreement with simulations of the nucleation rates that showed that most of the oligomeric species are formed around the half-time of the aggregation reaction with the formation of these species being delayed upon addition of Netoglitazone.

Next, an ELISA was carried out using an oligomer-specific antibody, allowing a direct measurement of the concentration of Aβ42 oligomers formed by aggregation reactions in the absence and presence of Netoglitazone. The results, shown in FIG. 1p, demonstrate a significant reduction in the Aβ42 oligomer concentration in the presence of Netoglitazone. As predicted by the kinetic studies, this further confirms that Netoglitazone is able to effectively suppress the aggregation of Aβ42.

### Example 6—Netoglitazone Rescues the Toxicity Induced by the Aggregation of the Aβ Peptide and Decreases the Plaques Load in a C. elegans Model (GMC101) of AD

In order to further confirm the inhibition of Aβ aggregation that is observed in vitro, the effects of Netoglitazone were tested using a well-known model of AD (GMC101). In this model the 42-residue isoform of the human Aβ peptide is over expressed in the big muscle cells of C. elegans worms and this leads to age dependent protein aggregation and consequent muscular paralysis.

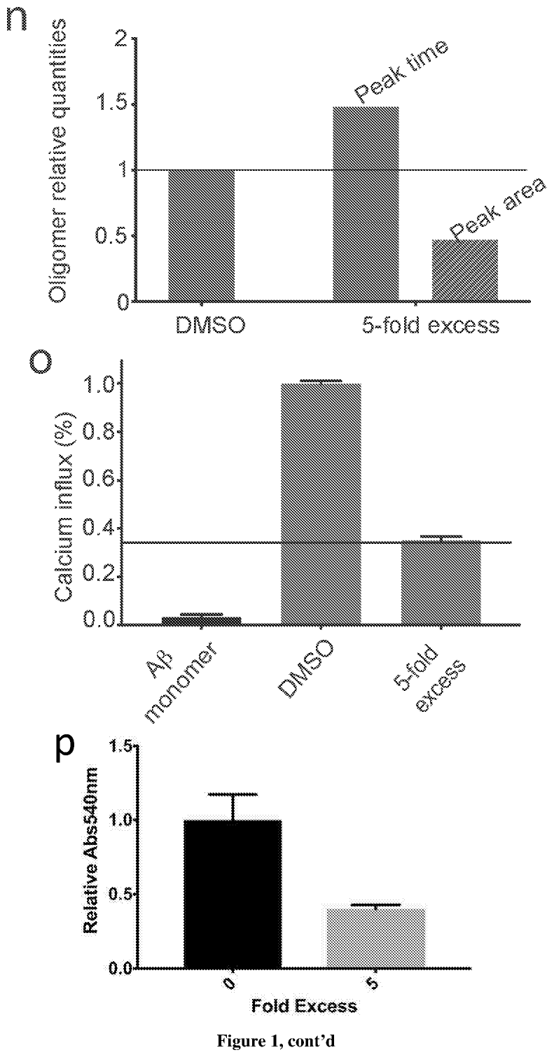

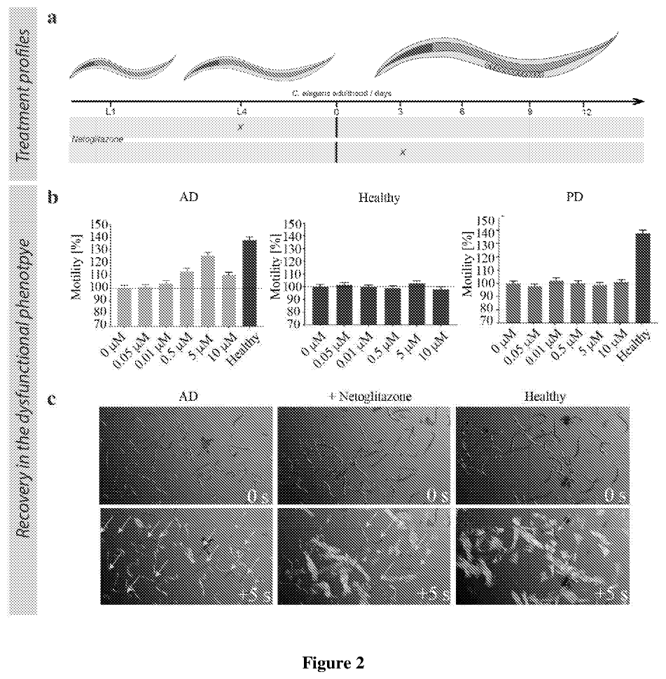

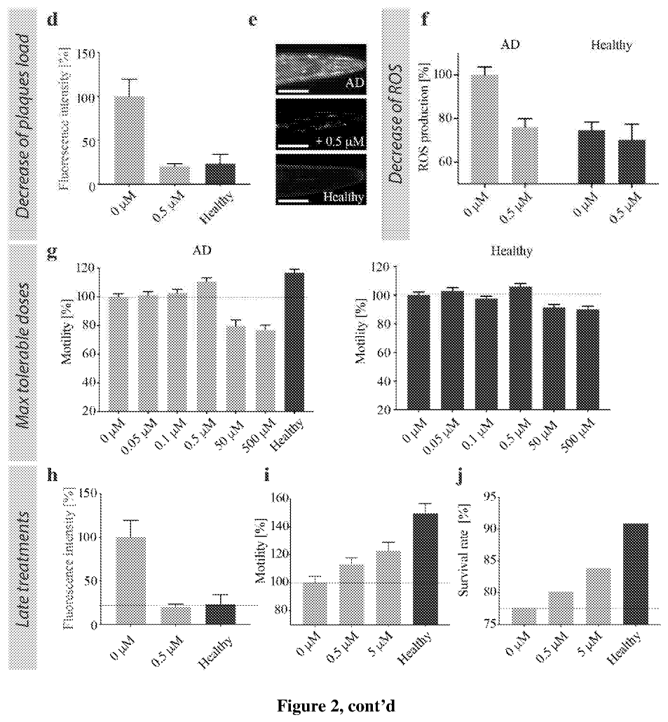

A treatment regime was at first defined by administering Netoglitazone at the last larval stage L4 (i. e. before the onset of the paralysis) as shown in FIG. 2a and then the mobility of the AD worms with the WF-NTP platform was screened at different ages of adulthood. The best protective effect was found to be observed at D3 of adulthood for a concentration range between 0.5-5 μM, as shown in FIGS. 2b and 2c. In order to further confirm the specificity of the observed effect in vivo, the same concentration range of Netoglitazone was administered to PD and healthy worms and in both cases the effect was found to be negligible compared to the observed effect in AD worms. This can be seen in FIGS. 2b and 2c.

As a next step, the effect of Netoglitazone on the aggregation profile of the Aβ peptide in the worms was investigated. By using the amyloid specific dye NIAD-4, it was possible to stain for the plaques load in living AD worms. It was observed that the administration of 0.5 μM of Netoglitazone could significantly decrease the plaques load in AD worms, as shown in FIGS. 2d and 2e.

The effect of Netoglitazone on the worm metabolic activities was investigated. Specifically, the levels of ROS production that are up-regulated in AD animals were measured compared to healthy controls, and it was observed that Netoglitazone significantly decreased the levels of oxidative species, as shown in FIG. 2f. Note that the maximum tolerable dose of Netoglitazone in AD worms was determined to be less than 50 μM, as shown in FIG. 2g.

The administration of Netoglitazone at L4 would in theory correspond to a preventative treatment since at the larval stages, no protein aggregates have been formed. This correlates extremely well with the in vitro studies where Netoglitazone was able to inhibit significantly the primary pathways. Given that Netoglitazone was also able to decrease the rate of surface-catalysed secondary nucleation and hence block the catalytic cycle of the aggregate proliferation, an assessment of this effect in vivo was sought. Netoglitazone was administered at D3 of adulthood, a scenario where protein aggregates have already formed and a dysfunction of the phenotype in AD animals can already be observed. Consequently, any possible effect of the drug would be ascribed to a therapeutic intervention by blocking the catalytic cycle of the aggregation inside the worms. Interestingly, in agreement with in vitro studies, it was found that this dosing regime also led to a significant decrease of the plaques load at D6 and an increase of the worm's mobility and survival rate, thus suggesting that Netoglitazone can affect secondary nucleation processes in vivo as well as in vitro. These results are shown in FIGS. 2h, 2i and 2j.

### Example 7—Other Glitazone Compounds in the Inhibition of Aβ Aggregation

Aβ42 fibril formation was monitored using fluorescence intensity in vitro using a 2 μM Aβ42 sample in the presence of Ciglitazone, Englitazone, Darglitazone, Troglitazone, Pioglitazone, Rosiglitazone, Rivoglitazone, Balaglitazone and Mitoglitazone, respectively, in the same manner as in Example 1.

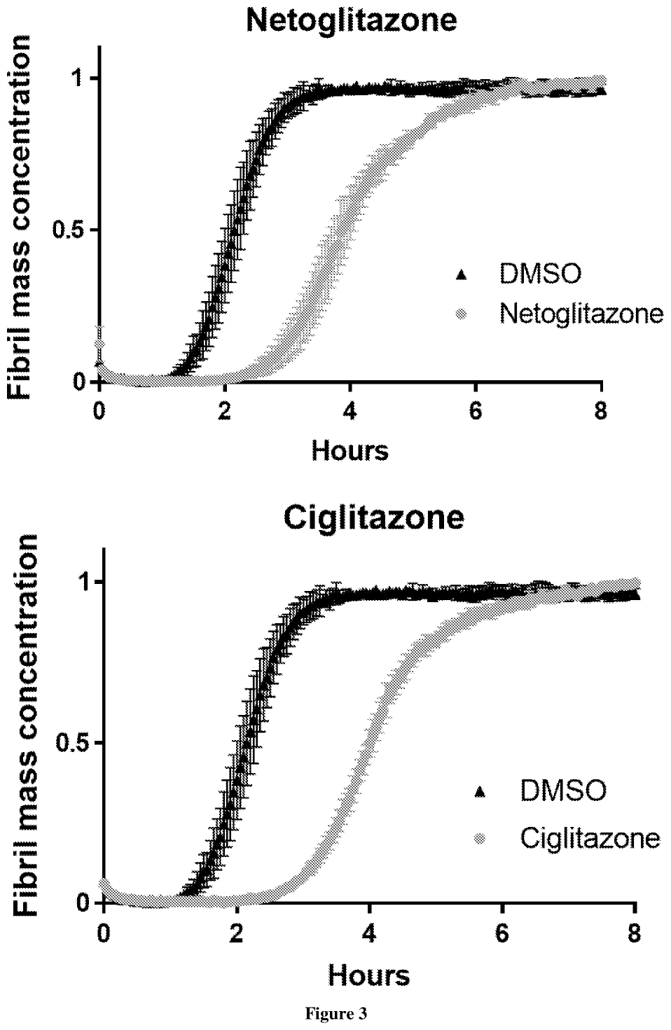

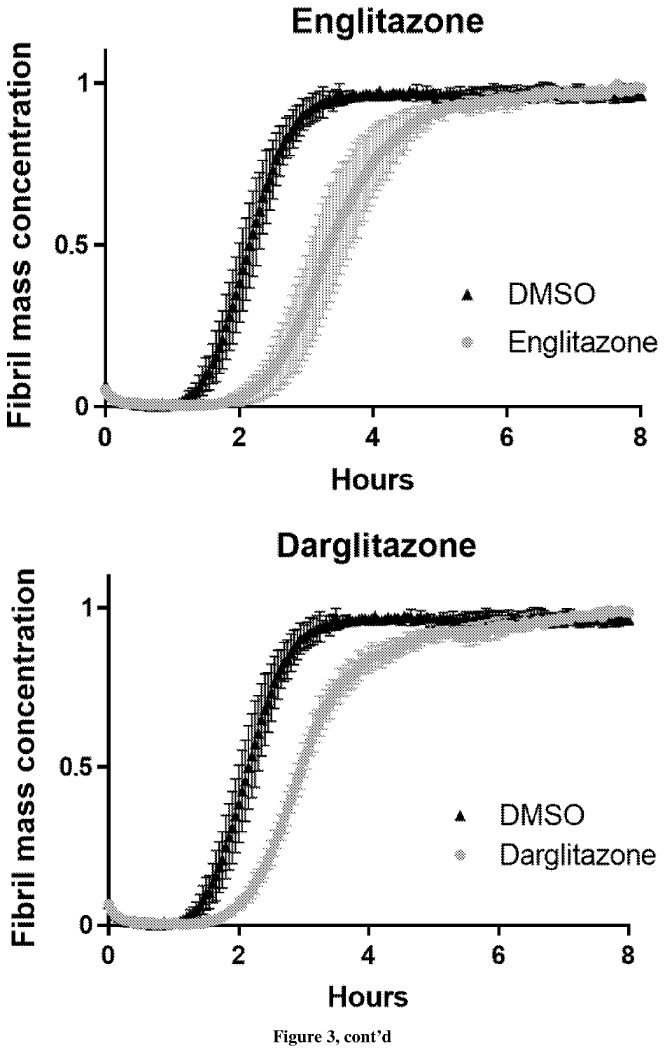

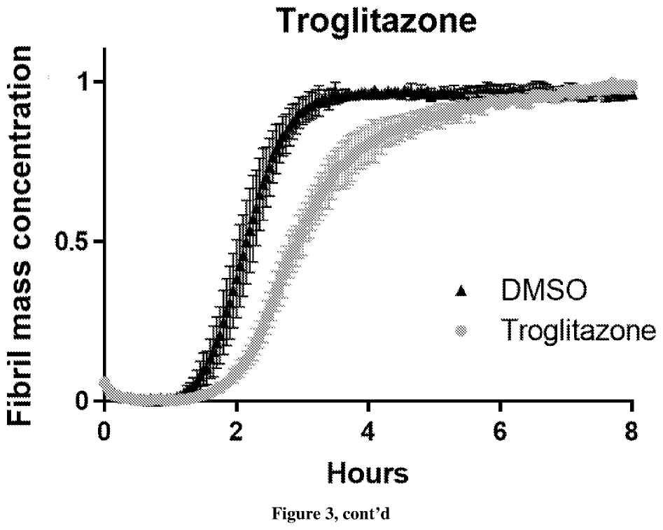

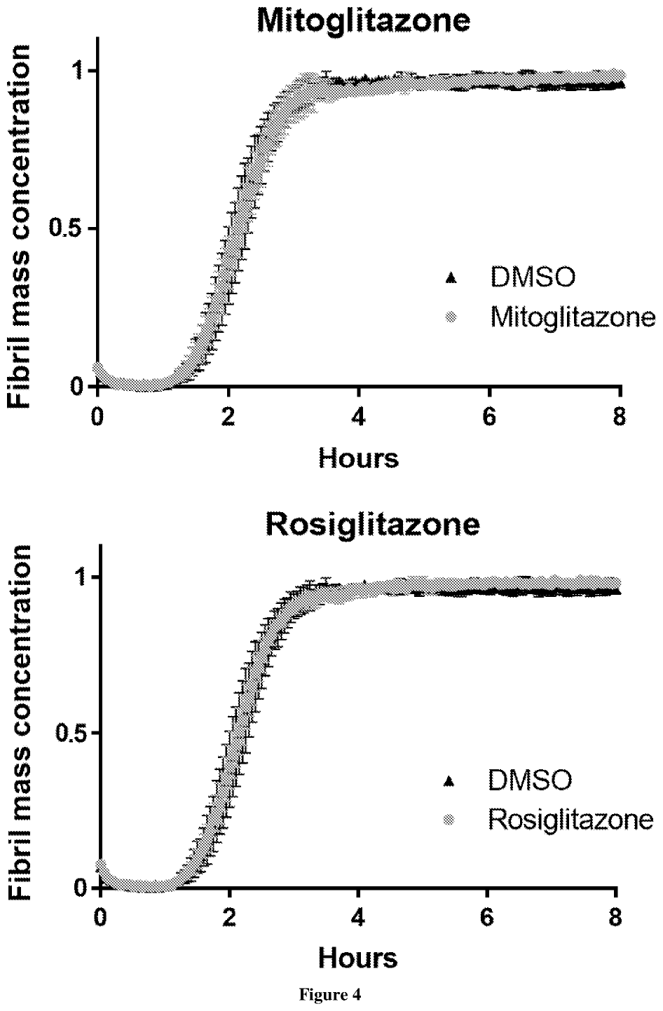

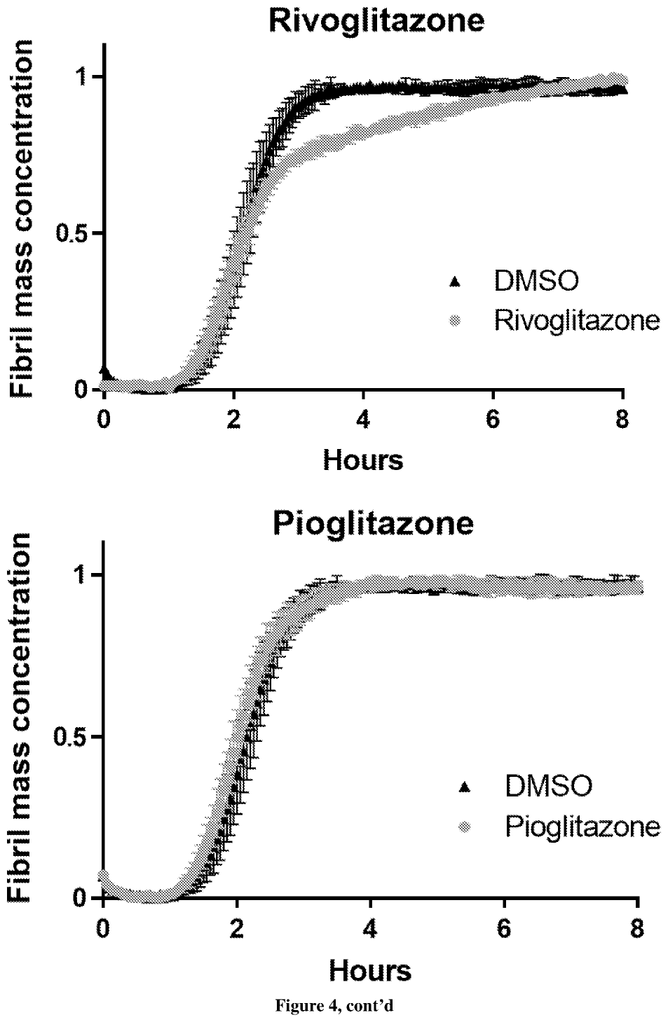

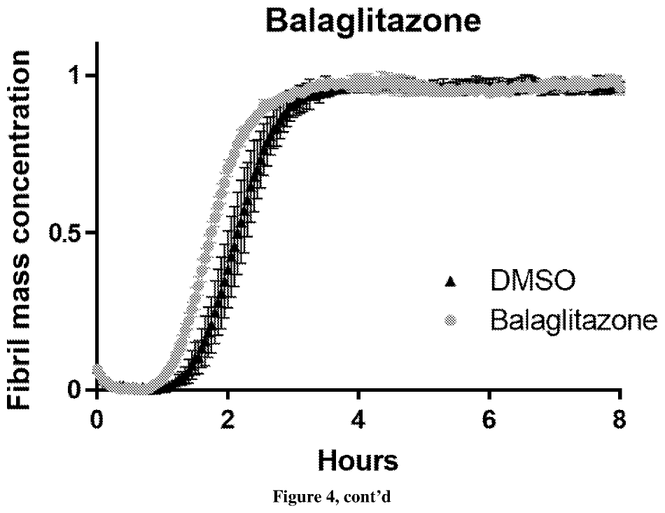

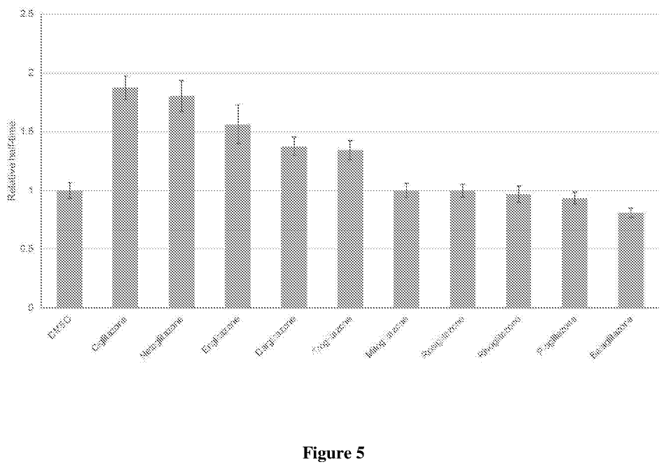

Ciglitazone, Englitazone, Darglitazone and Troglitazone were observed to delay Aβ42 aggregation. In particular, Ciglitazone and Englitazone significantly delayed aggregation. This can be seen in FIGS. 3 and 5. In the presence of Pioglitazone, Rosiglitazone, Rivoglitazone, Balaglitazone and Mitoglitazone at 5× drug:protein concentration, little to no delay of Aβ42 aggregation was observed, as shown in FIGS. 4 and 5.

### Example 8—the Effect of Netoglitazone on the Chemotaxis Index and Motility of Aβ_{1-42Neur} Worms and the Motility of Aβ₃₋₄₂::GF_{PMuscular} Worms

Further experiments with an additional C. elegans model were carried out, using Aβ_{1-42Neur} worms, which exhibit pan-neuronal expression of Aβ peptides. Netoglitazone was administered at concentrations ranging from 0.05 to 500 μM in 1% DMSO. As controls, plates seeded only with 1% DMSO were used.

Automated motility assays were carried out and the movements of the animals recorded. As shown in FIG. 6B, the results demonstrate that Netoglitazone significantly improves the motility of Aβ_{1-42Neur} worms when compared to untreated worms.

Chemotaxis assays were also carried out as shown in FIG. 6C, using Aβ_{1-42Neur} worms and wild-type healthy worms incubated with or without 5 μM Netoglitazone. As shown in FIG. 6A, the chemotaxis index was significantly improved in Aβ_{1-42Neur} worms treated with Netoglitazone when compared to untreated worms.

Motility experiments were also carried out with Aβ₃₋₄₂::GF_{PMuscular} worms. As shown in FIG. 6D, the results demonstrate that Netoglitazone also significantly improves the motility of this strain when compared to untreated worms.

### Example 9—Pharmacokinetic Studies in a Mouse Model

The pharmacokinetic profile was assessed in male, Swiss Albino mice in a discrete study using a single per oral dose of Netoglitazone (11.5 mg/kg, 30 μmoles/kg) formulated as a solution in 10% NMP/90% Solutol (as a 20% v/v solution in PBS). The dosing concentration was 2.3 mg/mL with a dosing volume of 5.0 mL/kg. Data were derived from the average of 3 animals per timepoint (terminal sampling). Plasma samples were collected at 0.25, 0.50, 1.0, 2.0, 4.0, 6.0, 8.0 and 24 hours by protein precipitation method with acetonitrile. Brain samples were collected at 6.0 and 24 hours by brain homogenisation and protein precipitation method with acetonitrile. Cerebrospinal fluid (CSF) samples were collected at 6.0 and 24 hours by protein precipitation method with acetonitrile. Samples were analysed using UHPLC with TOF mass spectrometry using electrospray ionisation.

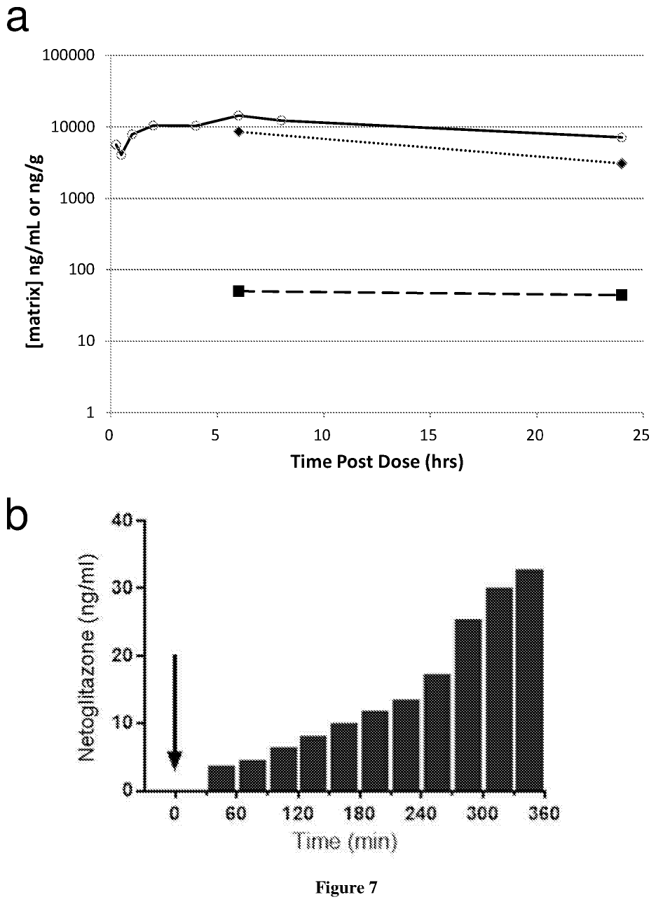

This pharmacokinetic study showed good plasma levels peaking at (Tₘₐₓ) 6 hours (Cₘₐₓ 14,393 ng/mL) with an estimated half-life of −19 hours, as shown in FIG. 7a. Total brain levels at 6 hours were 8542 ng/g thus confirming that Netoglitazone has good brain penetration across the blood brain barrier. The drug was also detected in CSF at 6 hours at levels of 50 ng/mL. CSF is a compartment of the CNS which is often used as a surrogate of CNS free drug levels due to the low levels of circulatory proteins and low abundance of lipid brain tissues. To access the CSF drugs typically need to cross the blood brain barrier. The CSF is in equilibrium with the CNS interstitial fluid (ISF) being separated by an epithelial layer. This data suggests that oral dosing of Netoglitazone can result in pharmacologically relevant free drug levels in the CNS.

To further assess the free drug exposure in the CNS following dosing of Netoglitazone an in vivo surgical microdialysis time course study was performed. Microdialysis is a minimally-invasive sampling technique that is used for continuous measurement of free, unbound analyte concentrations in the extracellular fluid of virtually any tissue (e.g. ISF in the brain). The microdialysis technique requires the insertion of a small microdialysis catheter (also referred to as microdialysis probe) into the tissue of interest. The microdialysis probe is designed to mimic a blood capillary and consists of a shaft with a semipermeable hollow fiber membrane at its tip, which is connected to inlet and outlet tubing. The probe is continuously perfused with an aqueous solution (perfusate) that closely resembles the (ionic) composition of the surrounding tissue fluid at a low flow rate of approximately 0.1-5 μL/min. Once inserted into the tissue or (body)fluid of interest, small solutes can cross the semipermeable membrane by passive diffusion. The direction of the analyte flow is determined by the respective concentration gradient and allows the usage of microdialysis probes as sampling as well as delivery tools. The solution leaving the probe (dialysate) is collected at certain time intervals for analysis. It is widely recognised as the ‘gold standard’ technique for measuring free drug levels in the CNS.

Briefly, 3 male C57BL/6 mice (18 weeks old) were surgically prepared with one cannula in the brain to allow for microdialysis sampling from the striatum. Animals were allowed one day to recover and then habituated to the microdialysis cages overnight. On the study day, a microdialysis probe was inserted through the implanted cannula. After 1 hour stabilisation, a pre-administration sample was collected, the animal was dosed with Netoglitazone (po, 15 mg/kg) formulated as a solution in 10% NMP/90% Solutol (as a 20% v/v solution in PBS) and samples were collected for 6 hours as detailed in Table 1 and FIG. 7b. At the end of the experiment animals were anaesthetised and terminal blood and brain samples collected.

Table 1 shows the levels of Netoglitazone in microdialysates from mouse striatum as ng/ml and ng/ml corrected for recovery (0.11) after 15 mg/kg dose.

TABLE 1

| Time after PO | Sample | | Netoglitazone (ng/ml) |
|---|---|---|---|
| dose (min) | ID | Netoglitazone (ng/ml) | corrected for recovery |
| −30-0 | B1 | <LLOQ | <LLOQ |
| 0-30 | B2 | <LLOQ | <LLOQ |
| 30-60 | B3 | 0.4 | 3.6 |
| 60-90 | B4 | 0.5 | 4.5 |
| 90-120 | B5 | 0.7 | 6.4 |
| 120-150 | B6 | 0.9 | 8.2 |
| 150-180 | B7 | 1.1 | 10.0 |
| 180-210 | B8 | 1.3 | 11.8 |
| 210-240 | B9 | 1.5 | 13.6 |
| 240-270 | B10 | 1.9 | 17.3 |
| 270-300 | B11 | 2.8 | 25.5 |
| 300-330 | B12 | 3.3 | 30.0 |
| 330-360 | B13 | 3.6 | 32.7 |

Terminal plasma and brain samples were collected and analysed for levels of Netoglitazone to confirm the peripheral exposure and total brain exposure at shortly after 6 hours (approximately Tₘₐₓ). This data is comparable with previous PK data and is summarized in Table 2. Table 2 shows terminal plasma and whole brain levels of Netoglitazone from microdialysis study mice collected post study.

TABLE 2

| Concentration in | Concentration in | |
|---|---|---|
| plasma (ng/ml) | brain (ng/g) | Brain:plasma ratio |
| 13266 | 10278 | 0.8 |

These data show that Netoglitazone readily crosses the blood-brain barrier after oral administration (15 mg/kg) and could be detected in microdialysate from fraction 30-60 min post administration. Levels in the ISF increased steadily up to an estimated concentration of 32.7 ng/ml (corrected for compound recovery) at fraction 330-360 min post administration. The temporal profile suggests that Tₘₐₓ may not have been achieved during collection time and that this data represents a conservative estimate of free drug Cₘₐₓ. Brain and plasma levels were 10278 ng/g and 13266 ng/ml respectively which are in agreement with previous studies.

### Example 10—Netoglitazone Shows Efficacy in a Mouse Model

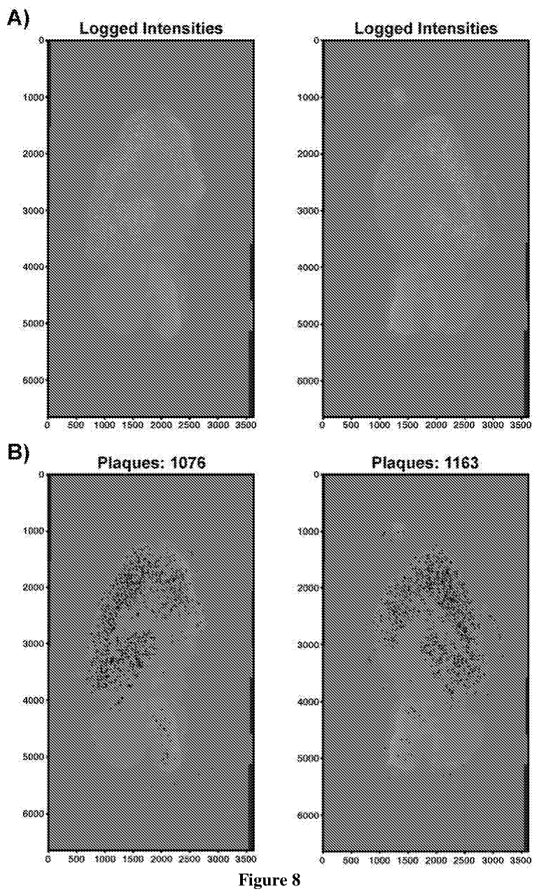

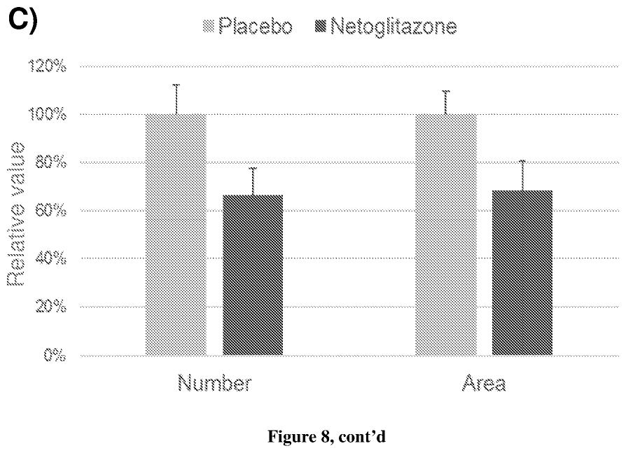

The efficacy of Netoglitazone in vivo was investigated using APPPS1 transgenic mice dosed once daily with either condensed milk and Netoglitazone or, for the placebo group, condensed milk only. At 150 days old, after 90 days of dosing, the mice were euthanized, perfused with a hydrogel solution and their brains analysed using the clearing and imaging methods and custom-coded software as discussed above. Images were produced of approximately 2500 two-dimensional slices per brain hemisphere and each slice was digitally analysed as shown in FIGS. 8A and 8B to quantify the total number and area of Aβ plaques across the entire brain. The results are shown in FIG. 8C, which shows a decrease in the relative number of Aβ plaques in the mice dosed with Netoglitazone when compared with the placebo mice.

1. A thiazolidinedione or rhodanine compound or a pharmaceutically acceptable salt, tautomer, solvate, hydrate, prodrug, derivative, stereoisomer, analog or isotopically labelled derivative thereof, for use in the treatment and/or prevention of a protein misfolding disease, wherein said compound is not Pioglitazone, Rosiglitazone, Rivoglitazone, Balaglitazone or Mitoglitazone.

2. A thiazolidinedione or rhodanine compound or a pharmaceutically acceptable salt, tautomer, solvate, hydrate, prodrug, derivative, stereoisomer, analog or isotopically labelled derivative thereof, for use in the treatment and/or prevention of a protein misfolding disease, wherein said compound comprises, at opposite ends of the molecule, a primary terminal group which is a thiazolidinedione or rhodanine group and a secondary terminal group which is not (i) a 5- to 10-membered partially unsaturated heterocyclyl group containing one or more nitrogen heteroatoms in the ring, or (ii) a 5- to 10-membered heteroaryl group containing one or more nitrogen heteroatoms in the ring.

3. A compound for use according to claim 1 or claim 2, wherein the compound is a compound of formula (I), or a pharmaceutically acceptable salt, tautomer, solvate, hydrate, prodrug, derivative, stereoisomer, analog or isotopically labelled derivative thereof: 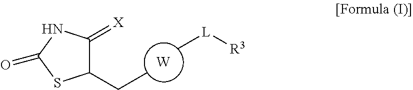
wherein:
X represents O or S;
W represents a benzene, naphthalene, benzodihydropyran or benzopyran ring, which is optionally further substituted;
L represents a linker group which comprises an alkylene group optionally comprising (i) one or more heteroatoms and/or carbonyl groups; and/or (ii) a 5- to 10-membered saturated or unsaturated heterocyclic group which is optionally substituted; and
R³ represents an optionally substituted C₆ to C₁₀ aryl group, optionally substituted C₅ to C₁₀ carbocyclyl group, optionally substituted 5- to 10-membered saturated heterocyclyl group, optionally substituted 5- to 10-membered partially unsaturated heterocyclyl group which does not contain a nitrogen heteroatom in the ring, or optionally substituted 5- to 10-membered heteroaryl group which does not contain a nitrogen heteroatom in the ring.

4. A compound for use according to claim 3, wherein the compound is a compound of formula (IA), or a pharmaceutically acceptable salt, tautomer, solvate, hydrate, prodrug, derivative, stereoisomer, analog or isotopically labelled derivative thereof: 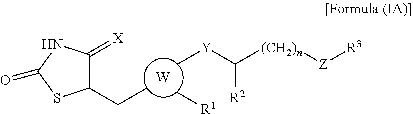
wherein:
X represents O or S;
W represents a benzene or naphthalene ring, which is optionally further substituted;
Y represents O or a carbonyl C(O) group;
R¹ and R² are the same or different and each independently represent hydrogen or a substituted or unsubstituted C₁ to C₄ alkyl group; or
R¹ and R² are linked to form a 5- to 7-membered aryl, carbocyclyl or heterocyclyl ring, which is optionally further substituted;
n is an integer of from 0 to 2;
Z represents a bond or a 5- to 10-membered saturated or unsaturated heterocyclic group which is optionally substituted; and
R³ represents an optionally substituted C₆ to C₁₀ aryl group, optionally substituted C₅ to C₁₀ carbocyclyl group or optionally substituted heterocyclyl group selected from pyranyl, dihydropyranyl, dihydrofuranyl, dihydrobenzofuranyl, dihydroisobenzofuranyl, benzopyranyl, dihydrobenzopyranyl, furanyl and benzofuranyl.

5. A compound for use according to claim 4, wherein:
X represents O;
W represents a benzene or naphthalene ring;
Y represents O;
R¹ and R² each independently represent hydrogen; or
R¹ and R² are linked to form, together with W, a benzopyran or benzodihydropyran ring; and
n is 0 or 1.

6. A compound for use according to any one of claims 3 to 5, wherein the compound is a compound of Formula (II) or (III), or a pharmaceutically acceptable salt, tautomer, solvate, hydrate, prodrug, derivative, stereoisomer, analog or isotopically labelled derivative thereof, 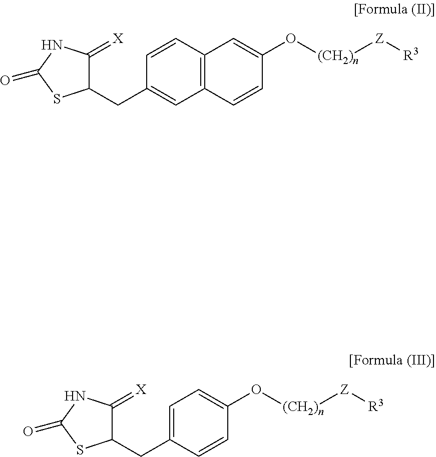
wherein:
n is 1 or 2; and
the other chemical groups are as defined in any one of claims 3 to 5.

7. A compound for use according to any one of claims 4 to 6, wherein:
Z represents a bond.

8. A compound for use according to any one of claims 3 to 7, wherein:
X represents O.

9. A compound for use according to any one of claims 3 to 8, wherein:
R³ represents a C₆ to C₁₀ aryl group or a C₅ to C₁₀ carbocyclyl group, optionally substituted by one or more hydroxyl, halogen and/or C₁ to C₄ alkyl groups.

10. A compound for use according to any one of claims 1 to 4, wherein said compound is Netoglitazone, Ciglitazone, Englitazone, Darglitazone or Troglitazone, or a pharmaceutically acceptable salt, tautomer, solvate, hydrate, prodrug, derivative, stereoisomer, analog or isotopically labelled derivative thereof.

11. A compound for use according to claim 10, wherein said compound is Netoglitazone, or a pharmaceutically acceptable salt, tautomer, solvate, hydrate, prodrug, derivative, stereoisomer, analog or isotopically labelled derivative thereof.

12. A compound for use according to any one of claims 1 to 11, for use in treating, preventing or inhibiting the formation, deposition, accumulation, or persistence of oligomers, fibrils, aggregates and/or plaques of proteins and/or peptides.

13. A compound for use according to claim 12, for use in treating, preventing or inhibiting the formation, deposition, accumulation, or persistence of amyloid (3 oligomers, fibrils, aggregates and/or plaques.

14. A compound for use according to any one of claims 1 to 13, wherein the protein misfolding disease is associated with misfolding of the amyloid-β peptide.

15. A compound for use according to any one of claims 1 to 14, wherein the protein misfolding disease is selected from amyloidosis, tauopathies, prion diseases (including Creutzfeld-Jakob disease and spongiform encephalopathies), neurodegenerative disease, Down syndrome, and/or cystic fibrosis.

16. A compound for use according to claim 15, wherein the protein misfolding disease is a neurodegenerative disease.

17. A compound for use according to claim 16, wherein the neurodegenerative disease is selected from dementia, mild cognitive impairment (MCI), Parkinson's disease, polyglutamine diseases (such as Huntington's disease) and/or amyotrophic lateral sclerosis (ALS).

18. A compound for use according to claim 17, wherein the dementia is selected from Alzheimer's disease, dementia with Lewy Bodies, frontotemporal dementia, familial dementia and/or progressive supranuclear palsy (PSP).

19. A compound for use according to claim 13 or claim 14, wherein the protein misfolding disease is selected from Alzheimer's disease, cerebral amyloid-β angiopathy, inclusion body myositis and/or Down's syndrome.

20. A compound for use according to any one of claims 1 to 19, wherein the protein misfolding disease is Alzheimer's disease.

21. A compound as defined in any one of claims 1 to 11, or a pharmaceutically acceptable salt, tautomer, solvate, hydrate, prodrug, derivative, stereoisomer, analog or isotopically labelled derivative thereof, for use in the treatment and/or prevention of a neurodegenerative disease.

22. A compound for use according to claim 21, wherein the neurodegenerative disease is selected from dementia, mild cognitive impairment (MCI), Parkinson's disease, polyglutamine diseases (such as Huntington's disease) and/or amyotrophic lateral sclerosis (ALS).

23. A compound for use according to claim 22, wherein the dementia is selected from Alzheimer's disease, dementia with Lewy Bodies, frontotemporal dementia, familial dementia and/or progressive supranuclear palsy (PSP).

24. A compound for use according to claim 23, wherein the dementia is Alzheimer's disease.

25. A compound for use according to claim 20 or claim 24, wherein the Alzheimer's disease is stage one, stage two or stage three Alzheimer's disease according to the Reisberg scale.

26. A compound for use according to any one of claims 1 to 25, for use in the treatment of a patient which has been diagnosed with, or is at risk of developing, Alzheimer's disease.

27. A compound for use according to any one of claims 1 to 26, wherein the patient has been diagnosed with mild cognitive impairment (MCI).

28. A compound for use according to claim 26 or 27, wherein the patient has a family history of Alzheimer's disease.

29. A pharmaceutical composition comprising a compound as defined in any one of claims 1 to 11 or a pharmaceutically acceptable salt, tautomer, solvate, hydrate, prodrug, derivative, stereoisomer, analog or isotopically labelled derivative thereof, for use in the treatment and/or prevention of a protein misfolding disease and/or a neurodegenerative disease.

30. A pharmaceutical composition for use according to claim 29, for use in the treatment and/or prevention of a protein misfolding disease and/or a neurodegenerative disease as defined in any one of claims 12 to 28.

31. A pharmaceutical composition for use according to claim 29 or claim 30, wherein the composition further comprises one or more additional pharmaceutically active agents.

32. A pharmaceutical composition for use according to claim 31, wherein the additional pharmaceutically active agent(s) are suitable for the treatment and/or prevention of a protein misfolding disease and/or a neurodegenerative disease.

33. A pharmaceutical composition for use according to claim 31 or claim 32, wherein the compound as defined in any one of claims 1 to 11 and the additional pharmaceutically active agent(s) are formulated for separate, concurrent, simultaneous or successive administration.

34. A pharmaceutical composition for use according to any one of claims 29 to 33, wherein the composition is formulated to improve penetration of the compound as defined in any one of claims 1 to 11 into the brain.

35. A composition for use according to claim 34, wherein the composition comprises nanoparticle carriers based on polymers, lipids, protein capsules or combinations thereof.

36. A kit comprising a compound as defined in any one of claims 1 to 11, or a pharmaceutically acceptable salt, tautomer, solvate, hydrate, prodrug, derivative, stereoisomer, analog or isotopically labelled derivative thereof, or a composition as defined in claim 29, for use in the treatment and/or prevention of a protein misfolding disease and/or a neurodegenerative disease.

37. The kit according to claim 36, wherein the kit further comprises, in admixture or in separate containers, an additional pharmaceutically active agent(s) as defined in claim 31 or claim 32.

38. A method of treating and/or preventing a protein misfolding disease and/or a neurodegenerative disease in a patient which comprises administering to said patient an effective amount of a compound as defined in any one of claims 1 to 11, or a pharmaceutically acceptable salt, tautomer, solvate, hydrate, prodrug, derivative, stereoisomer, analog or isotopically labelled derivative thereof.

39. A method according to claim 38, wherein the protein misfolding disease and/or neurodegenerative disease is as defined in any one of claims 12 to 28.

40. A method according to claim 39, wherein the protein misfolding disease and/or neurodegenerative disease is Alzheimer's disease.

41. Use of a compound as defined in any one of claims 1 to 11, or a pharmaceutically acceptable salt, tautomer, solvate, hydrate, prodrug, derivative, stereoisomer, analog or isotopically labelled derivative thereof, in the manufacture of a medicament for the treatment and/or prevention of a protein misfolding disease and/or a neurodegenerative disease.

42. Use according to claim 41, wherein the protein misfolding disease and/or neurodegenerative disease is as defined in any one of claims 12 to 28.

43. Use according to claim 42, wherein in the protein misfolding disease and/or neurodegenerative disease is Alzheimer's disease.
